(19) United States
(12) Patent Application Publication
(10) Pub. No.: US 2026/0317825 A1
(43) Pub. Date: Oct. 15, 2026

(54) Multi-Omic Approach for Assessing Systemic Lupus Erythematosus Heterogeneity in Treatment Response

(71) Applicant: Oklahoma Medical Research Foundation, Oklahoma City, OK (US)

(72) Inventors: Joel Guthridge, Oklahoma City, OK (US); Judith Ann James, Edmond, OK (US); Joan Merrill, Oklahoma City, OK (US); Miles Smith, Oklahoma City, OK (US)

(21) Appl. No.: 19/485,905

(22) PCT Filed: May 21, 2024
(86) PCT No.: PCT/US2024/030394

§ 371 Date: Nov. 19, 2025

Related U.S. Application Data
(60) Provisional application No. 63/503,995, filed on May 24, 2023.

(51) Int. Cl. C12Q 1/6883

(52) U.S. Cl. CPC C12Q 1/6883; C12Q 2600/106; C12Q 2600/154

(57) ABSTRACT

Provided herein are methods for treating lupus patients comprising determining whether a patient has a genetic predisposition to being responsive to the treatment with the inhibitor of CD80/86 immune cell costimulation by obtaining or having obtained a whole blood sample from the patient with lupus, separating the T cells, B cells, and monocytes, and performing or having performed a methylation assay on the genome of at least one of the T cells, B cells, or monocytes determining if the patient has hypomethylation at the one or more CpG biomarkers when compared to a subject that does not have lupus in the least one of the T cells, B cells, and monocytes, and administering the inhibitor of CD80/86 immune cell costimulation if the patient has hypomethylation of the one or more of the CpG biomarkers in the least one of the T cells, B cells, and monocytes.

### CROSS-REFERENCE TO RELATED APPLICATIONS

This application is a non-provisional patent application of and claims priority to U.S. provisional patent application Ser. No. 63/503,995 filed on May 24, 2023, the contents of which are incorporated by reference in its entirety.

### STATEMENT OF FEDERALLY FUNDED RESEARCH

This invention was made with government support under UM1 AI144292 awarded by the National Institutes of Health. The government has certain rights in the invention.

### TECHNICAL FIELD OF THE INVENTION

The present invention relates in general to the field of treatments for systemic lupus erythematosus, and more particularly, to a multi-omic approach to assess lupus heterogeneity in treatment response.

### INCORPORATION-BY-REFERENCE OF MATERIALS FILED ON COMPACT DISC

None.

### BACKGROUND OF THE INVENTION

Without limiting the scope of the invention, its background is described in connection with the detection of systemic lupus erythematosus.

One such patent is U.S. Pat. No. 11,644,473, entitled, “Serum Biomarkers For Predicting And Evaluating Response To TNF Inhibitor Therapy In Rheumatoid Arthritis Patients”, issued to Han, et al. These inventors are said to teach methods and kits for using serum biomarkers C-X-C Motif Chemokine Ligand 10 (CXCL10), C-X-C Motif Chemokine Ligand 13 (CXCL13), and/or soluble CD27 (sCD27), in predicting and evaluating therapeutic response to Tumor Necrosis Factor (TNF) inhibitor therapy in a patient in need thereof.

One such patent application is U.S. Patent Publication No. 20230142955, entitled, “Methods of Using A Multi-Analyte Approach For Diagnosis And Staging A Disease”, filed by Issadore, et al. These applicants are said to teach a method for determining or diagnosing a disease, classifying a stage of a disease, or treating a disease or methods for assessing the efficacy of a therapy for treating a disease based on the measurement and the computational analysis of various disease-specific biomarkers by measuring, in a processed sample from the subject, a set of circulating biomarkers comprising an extra-cellular vesicle (EV) miRNA, an EV mRNA, a circulating cell-free DNA, a circulating tumor DNA, and a protein biomarker specific for the disease or the condition; applying a machine learning algorithm on the set of circulating biomarkers to generate an output indicative of a disease or a condition state of the subject; determining whether the subject has the disease or the condition based upon the output so generated; and treating the subject as needed. The method involves obtaining both nucleic acids and proteins to make an assessment.

Despite these advances, a need remains for novel biomarkers, methods, and the selection of effective treatments for systemic lupus erythematosus.

### SUMMARY OF THE INVENTION

As embodied and broadly described herein, an aspect of the present disclosure relates to a method to select lupus patients that will be responsive to a treatment with an inhibitor of CD80/86 immune cell costimulation, comprising: determining whether a patient has a genetic predisposition to being responsive to the treatment with the inhibitor of CD80/86 immune cell costimulation by: obtaining or having obtained a whole blood sample from the patient with lupus; separating T cells, B cells, and monocytes; performing or having performed a methylation assay on a genome of at least one of the T cells, B cells, or monocytes to detect hypomethylation at one or more CpG biomarkers selected from; Cg04612171; Cg16733676; Cg02901522; or Cg11986743; determining if the patient has hypomethylation at the one or more CpG biomarkers when compared to a subject that does not have lupus in the at least one of the T cells, B cells, and monocytes, and administering the inhibitor of CD80/86 immune cell costimulation if the patient has hypomethylation of the one or more of the CpG biomarkers in the least one of the T cells, B cells, and monocytes; or discontinuing treatment with the inhibitor of CD80/86 immune cell costimulation if the patient does not have hypomethylation of the one or more of the CpG biomarkers in the least one of the T cells, B cells, and monocytes. In one aspect, the inhibitor of CD80/86 immune cell costimulation is Abatacept or Belatacept. In another aspect, the hypomethylation is detected for any 2, 3 or 4 of Cg04612171 and Cg16733676; Cg02901522; and Cg11986743. In another aspect, the hypomethylation is detected for any 2 or 3 of the T cells, B cells, and monocytes. In another aspect, the hypomethylation is detected for any 2 or 3 of the T cells, B cells, and monocytes and is detected for any 2, 3 or 4 of Cg04612171 and Cg16733676; Cg02901522; and Cg11986743. In another aspect, the hypomethylation is detected in T cells and there is an increase in T cell receptor (TCR) signaling genes, a decrease in T cell proliferation, or both. In another aspect, the hypomethylation is detected in monocytes and there is an increase in expression of genes in retinoic acid pathway genes. In another aspect, the hypomethylation is detected in B cells and there is a decrease in B cell receptor (BCR) activity, Fc gamma receptor IIB (FCγRIIB) activity, or both. In another aspect, the hypomethylation is detected by bisulfite sequencing, high-performance liquid chromatography-ultraviolet (HPLC-UV), liquid chromatography coupled with tandem mass spectrometry (LC-MS/MS), enzyme-linked immunosorbent assay (ELISA), long interspersed nuclear elements (LINE-1) and Pyrosequencing, amplification fragment length polymorphism (AFLP), restriction fragment length polymorphism (RFLP), luminometric methylation assay, bead-chip, bead-chip array, microarray, methyl-sensitive cut counting, or luminometric methylation. In another aspect, the hypomethylation of Cg04612171 and Cg16733676; Cg02901522; and Cg11986743, affects expression of Sine Oculis Binding Protein Homolog (SOBP), Solute Carrier Family 25 Member 24 (SLC25A24), Plasmacytoma Variant Translocation 1 (PVT1), or Beta-1,4-Galactosyltransferase 6 (B4GALT6), respectively. In another aspect, the lupus is systemic lupus erythematosus, Cutaneous lupus erythematosus, drug-induced lupus, or neonatal lupus. In another aspect, the method further comprises the step of monitoring a methylation status of the one or more CpG biomarkers at one or more points in time and based on a change in methylation discontinuing or restarting treatment with the inhibitor of CD80/86 immune cell costimulation. In another aspect, the method further comprises the step of monitoring a methylation status of the one or more CpG biomarkers at one or more points in time of a patient undergoing treatment with the inhibitor of CD80/86 immune cell costimulation and based on a change in methylation selecting an alternative treatment.

As embodied and broadly described herein, an aspect of the present disclosure relates to a method of treating lupus patients that will be responsive for a treatment with an inhibitor of CD80/86 immune cell costimulation, comprising: determining whether a patient has a genetic predisposition to being responsive to the treatment with the inhibitor of CD80/86 immune cell costimulation: obtaining or having obtained a whole blood sample from the patient with lupus; separating T cells, B cells, and monocytes; performing or having performed a methylation assay on a genome of at least one of the T cells, B cells, or monocytes to detect methylation at one or more CpG biomarkers selected from; Cg04612171; Cg16733676; Cg02901522; or Cg11986743; determining if the lupus patient has hypomethylation at the one or more CpG biomarkers when compared to a subject that does not have lupus in the at least one of the T cells, B cells, and monocytes, wherein the lupus is systemic lupus erythematosus, Cutaneous lupus erythematosus, drug-induced lupus, or neonatal lupus; and administering the inhibitor of CD80/86 immune cell costimulation if the patient has hypomethylation of the one or more of the CpG biomarkers in the least one of the T cells, B cells, and monocytes; or discontinuing treatment with the inhibitor of CD80/86 immune cell costimulation if the patient does not have hypomethylation of the one or more of the CpG biomarkers in the least one of the T cells, B cells, and monocytes and administering the patient with at least one of NSAIDs, corticosteroids, immunosuppressants, hydroxychloroquine, or methotrexate. In another aspect, the inhibitor of CD80/86 immune cell costimulation is Abatacept or Belatacept. In another aspect, the hypomethylation is detected for any 2, 3 or 4 of Cg04612171 and Cg16733676; Cg02901522; and Cg11986743. In another aspect, the hypomethylation is detected for any 2 or 3 of the T cells, B cells, and monocytes. In another aspect, the hypomethylation is detected for any 2 or 3 of the T cells, B cells, and monocytes and is detected for any 2, 3 or 4 of Cg04612171 and Cg16733676; Cg02901522; and Cg11986743. In another aspect, the hypomethylation is detected by bisulfite sequencing, high-performance liquid chromatography-ultraviolet (HPLC-UV), liquid chromatography coupled with tandem mass spectrometry (LC-MS/MS), enzyme-linked immunosorbent assay (ELISA), long interspersed nuclear elements (LINE-1) and Pyrosequencing, amplification fragment length polymorphism (AFLP), restriction fragment length polymorphism (RFLP), luminometric methylation assay, bead-chip, bead-chip array, microarray, methyl-sensitive cut counting, or luminometric methylation. In another aspect, the method further comprises the step of monitoring a methylation status of the one or more CpG biomarkers at one or more points in time and based on a change in methylation discontinuing or restarting treatment with the inhibitor of CD80/86 immune cell costimulation. In another aspect, the method further comprises the step of monitoring a methylation status of the one or more CpG biomarkers at one or more points in time of a patient undergoing treatment with the inhibitor of CD80/86 immune cell costimulation and based on a change in methylation selecting an alternative treatment.

As embodied and broadly described herein, an aspect of the present disclosure relates to a CTLA4/T cell activation methylation signature predicting responsiveness to an inhibitor of CD80/86 immune cell costimulation, the signature comprising: hypomethylation in a genome of at least one of T cells, B cells, or monocytes in one or more CpG biomarkers selected from; Cg04612171; Cg16733676; Cg02901522; or Cg11986743; wherein the hypomethylation at the one or more CpG biomarkers is compared to a subject that does not have lupus in the at least one of the T cells, B cells, and monocytes. In one aspect, the inhibitor of CD80/86 immune cell costimulation is Abatacept or Belatacept. In another aspect, the hypomethylation is detected for any 2, 3 or 4 of Cg04612171 and Cg16733676; Cg02901522; and Cg11986743. In another aspect, the hypomethylation is detected for any 2 or 3 of the T cells, B cells, and monocytes. In another aspect, the hypomethylation is detected for any 2 or 3 of the T cells, B cells, and monocytes and is detected for any 2, 3 or 4 of Cg04612171 and Cg16733676; Cg02901522; and Cg11986743. In another aspect, the hypomethylation is detected in T cells and there is an increase in T cell receptor (TCR) signaling genes and a decrease in T cell proliferation. In another aspect, the hypomethylation is detected in monocytes and there is an increase in expression of genes in retinoic acid pathway genes. In another aspect, the hypomethylation is detected in B cells and there is a decrease in B cell receptor (BCR) activity, Fc gamma receptor IIB (FCγRIIB) activity. In another aspect, the hypomethylation is detected by bisulfite sequencing, high-performance liquid chromatography-ultraviolet (hypomethylation-UV), liquid chromatography coupled with tandem mass spectrometry (LC-MS/MS), enzyme-linked immunosorbent assay (ELISA), long interspersed nuclear elements (LINE-1) and Pyrosequencing, amplification fragment length polymorphism (AFLP), restriction fragment length polymorphism (RFLP), luminometric methylation assay, bead-chip, bead-chip array, microarray, methyl-sensitive cut counting, or luminometric methylation. In another aspect, the hypomethylation of Cg04612171 and Cg16733676; Cg02901522; and Cg11986743, affects expression of Sine Oculis Binding Protein Homolog (SOBP), Solute Carrier Family 25 Member 24 (SLC25A24), Plasmacytoma Variant Translocation 1 (PVT1), or Beta-1,4-Galactosyltransferase 6 (B4GALT6), respectively.

As embodied and broadly described herein, an aspect of the present disclosure relates to a method for diagnosing lupus patients responsive to treatment with an inhibitor of CD80/86 immune cell costimulation, comprising: determining whether a patient has a genetic predisposition to being responsive to the treatment with the inhibitor of CD80/86 immune cell costimulation: obtaining or having obtained a whole blood sample from the patient with lupus; separating T cells, B cells, and monocytes; performing or having performed a methylation assay on a genome of at least one of the T cells, B cells, or monocytes to detect methylation at one or more CpG biomarkers selected from; Cg04612171; Cg16733676; Cg02901522; or Cg11986743; and determining if the lupus patient has hypomethylation at the one or more CpG biomarkers when compared to a subject that does not have lupus in the at least one of the T cells, B cells, and monocytes, wherein the lupus is systemic lupus erythematosus, Cutaneous lupus erythematosus, drug-induced lupus, or neonatal lupus, wherein patients with hypomethylation responsive to treatment with the inhibitor of CD80/86 immune cell costimulation. In one aspect, the lupus is systemic lupus erythematosus, Cutaneous lupus erythematosus, drug-induced lupus, or neonatal lupus. In another aspect, the method further comprises the step of monitoring a methylation status of the one or more CpG biomarkers at one or more points in time and based on a change in methylation discontinuing or restarting treatment with the inhibitor of CD80/86 immune cell costimulation. In another aspect, the method further comprises the step of monitoring a methylation status of the one or more CpG biomarkers at one or more points in time of a patient undergoing treatment with the inhibitor of CD80/86 immune cell costimulation and based on a change in methylation selecting an alternative treatment.

### BRIEF DESCRIPTION OF THE DRAWINGS

For a more complete understanding of the features and advantages of the present invention, reference is now made to the detailed description of the invention along with the accompanying figures and in which:

FIG. 1. Overview of total samples and study design. Flowchart depicting the overall systems biology methodology applied to the samples and processing implemented in this study. In brief, PBMCs and serum were collected from screening visits of abatacept arm patients from two placebo-controlled trials of abatacept, ABC (n=19) and ACCESS (n=40). Serum soluble mediators were assessed on a custom panel of 50 analytes. PBMCs were fractionated into major immune cell populations for parallel epigenomic and transcriptomic profiling. These data were integrated and collectively analyzed to develop a model predicting lupus response to abatacept at screening. ABT=abatacept; CTX=cyclophosphamide; R=responder; NR=non-responder.

FIG. 2. Comparison of cell type-specific methylation differences of top 4 performing CpGs by abatacept response. Violin dot plots of methylation differences (as beta values) by abatacept response of top 4 CpGs in each cell type. Red violins are for responders (R) and blue violins are for non-responders (NR). Asterisks indicate significance of Benjamini-Hochberg corrected p-value of Mann-Whitney U-Tests: *<0.05, **<0.01, ***<0.001, ****<0.0001.

FIG. 3. Clustering of SLE patients by methylation status of 4 CpGs predicting abatacept response. Heatmap of methylation status (as beta values) of top 4 CpGs in each cell type across all SLE patients. Each column represents a single patient, with each row representing CpG within a cell type (denoted by the first letter: t=T cells, b=B cells, m=monocytes). Dendrogram at top represents hierarchical clustering of SLE patients. Response to abatacept (Response), trial of origin (Study), ethnicity, and age are also denoted.

FIG. 4. Genomic regions near cg04612171. UCSC Genome Browser view of the genomic and regulatory context of near the cg04612171 CpG. Tracks show the location of the CpG of interest, GENCODE annotated transcripts, CpG islands, ENCODE predicted cis-regulatory elements, Encode activating histone acetylation, density of ReMap transcription factor binding sites, and common SNPs (MAF>1%) in dbSNP. Coordinates are presented from the hg38 build of the human genome.

FIG. 5. Methylation linkage in cg04612171 with nearby CpG sites. Linkage plot depicting linkage between methylation at the site of interest, cg04612171, and surrounding CpG sites as the coefficient of determination (R2) between the methylation M-values. Linkage was calculated to CpG sites within 10 kbp independently in each cell type assayed. Color of each point indicates annotated genetic region of each CpG site.

FIG. 6. Genomic regions near cg16733676. UCSC Genome Browser view of the genomic and regulatory context of near the cg16733676 CpG. Tracks show the location of the CpG of interest, GENCODE annotated transcripts, CpG islands, ENCODE predicted cis-regulatory elements, Encode activating histone acetylation, density of ReMap transcription factor binding sites, and common SNPs (MAF>1%) in dbSNP. Coordinates are presented from the hg38 build of the human genome.

FIG. 7. Methylation linkage in cg16733676 with nearby CpG sites. Linkage plot depicting linkage between methylation at the site of interest, cg16733676, and surrounding CpG sites as the coefficient of determination (R2) between the methylation M-values. Linkage was calculated to CpG sites within 10 kbp independently in each cell type assayed. Color of each point indicates annotated genetic region of each CpG site.

FIG. 8. Genomic regions near cg02901522. UCSC Genome Browser view of the genomic and regulatory context of near the cg02901522 CpG. Tracks show the location of the CpG of interest, GENCODE annotated transcripts, CpG islands, ENCODE predicted cis-regulatory elements, Encode activating histone acetylation, density of ReMap transcription factor binding sites, and common SNPs (MAF>1%) in dbSNP. Coordinates are presented from the hg38 build of the human genome.

FIG. 9. Methylation linkage in cg02901522 with nearby CpG sites. Linkage plot depicting linkage between methylation at the site of interest, cg02901522, and surrounding CpG sites as the coefficient of determination (R2) between the methylation M-values. Linkage was calculated to CpG sites within 25 kbp independently in each cell type assayed. Color of each point indicates annotated genetic region of each CpG site.

FIG. 10. Genomic regions near cg11986743. UCSC Genome Browser view of the genomic and regulatory context of near the cg11986743 CpG. Tracks show the location of the CpG of interest, GENCODE annotated transcripts, CpG islands, ENCODE predicted cis-regulatory elements, Encode activating histone acetylation, density of ReMap transcription factor binding sites, and common SNPs (MAF>1%) in dbSNP. Coordinates are presented from the hg38 build of the human genome.

FIG. 11. Methylation linkage in cg11986743 with nearby CpG sites. Linkage plot depicting linkage between methylation at the site of interest, cg11986743, and surrounding CpG sites as the coefficient of determination (R2) between the methylation M-values. Linkage was calculated to CpG sites within 50 kbp independently in each cell type assayed. Color of each point indicates annotated genetic region of each CpG site.

FIG. 12. Correlation of T cell RNA transcripts with parallel methylation at CpGs predictive of abatacept response. Volcano plots showing the results of correlation analysis of methylation at each of four predictive CpGs with parallel RNA-seq data in T cells. The slope of each correlated relationship (unit change in transcript per unit change in methylation) is plotted on the x-axis, and values on the y-axis represent negative log-transforms of p-values adjusted for multiple comparisons. Horizontal and vertical dashed lines indicate cutoffs for significance. Positive- and negative-associated transcripts passing these cutoffs are marked in red and green, respectively. The highest positive- and negative-associated transcripts are labeled on each plot. Correlation of B cell RNA transcripts with parallel methylation at CpGs predictive of abatacept response. Volcano plots showing the results of correlation analysis of methylation at each of four predictive CpGs with parallel RNA-seq data in B cells. The slope of each correlated relationship (unit change in transcript per unit change in methylation) is plotted on the x-axis, and values on the y-axis represent negative log-transforms of p-values adjusted for multiple comparisons. Horizontal and vertical dashed lines indicate cutoffs for significance. Positive- and negative-associated transcripts passing these cutoffs are marked in red and green, respectively. The highest positive- and negative-associated transcripts are labeled on each plot. Correlation of monocyte RNA transcripts with parallel methylation at CpGs predictive of abatacept response. Volcano plots showing the results of correlation analysis of methylation at each of four predictive CpGs with parallel RNA-seq data in monocytes. The slope of each correlated relationship (unit change in transcript per unit change in methylation) is plotted on the x-axis, and values on the y-axis represent negative log-transforms of p-values adjusted for multiple comparisons. Horizontal and vertical dashed lines indicate cutoffs for significance. Positive- and negative-associated transcripts passing these cutoffs are marked in red and green, respectively. The highest positive- and negative-associated transcripts are labeled on each plot.

FIG. 13. Ingenuity Canonical Pathway Analysis of T cell transcriptome correlations with methylation at predictive CpGs. Summary of most significant results comparing Canonical Pathway Analysis of by QIAGEN's Ingenuity® Pathway Analysis (IPA). The most significantly correlated T cell transcripts with methylation at each CpG of interest were input into IPA with previously noted cutoffs. Rows represent the most significant results across all analyses, and columns represent the correlations with each CpG site noted at the bottom. Color indicates the activation z-score, which represents a predicted change in pathway activity correlating with methylation.

FIG. 14. Ingenuity Canonical Pathway Analysis of B cell transcriptome correlations with methylation at 3 CpG sites of interest. Summary of most significant results comparing Canonical Pathway Analysis of by QIAGEN's Ingenuity® Pathway Analysis (IPA). The most significantly correlated B cell transcripts with methylation at each CpG of interest were input into IPA with previously noted cutoffs. Rows represent the most significant results across all analyses, and columns represent the correlations with each CpG site noted at the bottom. Color indicates the activation z-score, which represents a predicted change in pathway activity correlating with methylation.

FIG. 15. Ingenuity Canonical Pathway Analysis of monocyte transcriptome correlations with methylation at CpG sites of interest. Summary of most significant results comparing Canonical Pathway Analysis of by QIAGEN's Ingenuity® Pathway Analysis (IPA). The most significantly correlated monocyte transcripts with methylation at each CpG of interest were input into IPA with previously noted cutoffs. Rows represent the most significant results across all analyses, and columns represent the correlations with each CpG site noted at the bottom. Color indicates the activation z-score, which represents a predicted change in pathway activity correlating with methylation.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

Systemic Lupus Erythematosus (SLE) is a complex autoimmune disease often necessitating complex management. However, treatment options for patients are limited. As of 2023, federal regulators in the United States have approved six medications for the treatment of SLE. Existing regimens involve combinations of antimalarials, corticosteroids, and immunosuppressants depending on the clinical manifestations and the prescriber's preferences. While these treatments can be effective at easing symptoms, SLE patients still have increased morbidity and mortality due to drug toxicity and persistent disease activity^{1,2}. Despite recent successes of belimumab, anifrolumab and voclosporin, trials of over 30 promising agents over the past decades have resulted in disproportionate failure³⁻¹⁰. This high rate of investigational failure is almost certainly impacted by the vast heterogeneity of SLE. In particular, the simultaneous immunosuppressive and immunomodulating treatments commonly employed in SLE inevitably interact with investigational agents, resulting in high placebo response rates^{11,12}. This is superimposed on heterogeneity within the disease itself. Heritable and non-heritable factors contribute to diversity in SLE progression and outcomes¹³⁻¹⁸. The combination of these factors produces enough biological noise to ensure the failure of not only SLE clinical trials, but also investigations into mechanisms underlying disease.

Abatacept is an example of a biologic therapy that is a fusion protein of the cytotoxic T-lymphocyte associated protein 4 (CTLA4) and the fragment crystallizable (Fc) region of IgG. This medication targets the co-stimulation of lymphocytes by antigen presenting cells by disrupting the interaction of B7 molecules (CD80 and CD86) with CD28 and has received regulatory approval for the treatment of rheumatoid arthritis, psoriatic arthritis, and juvenile idiopathic arthritis. Since abatacept is predicted to induce tolerance in autoreactive T cells, several randomized phase II controlled trials have assessed the efficacy of abatacept in the treatment of SLE¹⁹⁻²¹. While, these trials have failed to meet their primary endpoints over placebo (standard of care), exploratory investigations and endpoints suggest that a subset of SLE patients may receive some benefit from abatacept treatment. Post hoc analysis of a phase IIb trial of abatacept in SLE showed that abatacept was superior to placebo in preventing arthritis flares and acute flares as defined by Physician Global Assessment (PGA)²⁰. Further analysis of whole blood transcriptomic data from this same trial showed that a patient cluster dominated by activated dendritic cell, plasma cell, and neutrophil signatures at baseline showed the greatest reduction in disease activity scores over a year²².

The present inventors used a systems biology approach using a multi-omic data analysis across immune subsets for a global view of the immune system. Such studies have highlighted the importance of DNA methylation in SLE²³, with T cells, in particular, demonstrating aberrant methylation profiles²⁴⁻²⁶. DNA methylation, the covalent addition of a CH₃-moiety to cytosine bases by DNA-methyltransferases, is one of a number of epigenetic modifications that can have profound impacts on subsequent transcription. Methylation of sites located within promoters and enhancers can block the accessibility of these motifs and prevent the binding of transcription factors and activation of genes in response to intra- and extracellular stimuli.

Previous genome-wide studies comparing SLE to healthy controls have revealed consistent hypomethylation of interferon-regulated genes extending across T cells²⁷, B cells, monocytes²⁸, and granulocytes²⁹, permitting a rapid response to interferon in most immune cells. Transcriptomic assays have also been employed to analyze the peripheral immune environment of SLE and identified new roles for interferon, erythropoiesis, myeloid cells, and mitochondrial activity in disease^{30,31}. Both of these molecular techniques have been previously applied to large cohorts examining either mixed immune populations in whole blood or isolated cell types of interest. Broad omics profiling has also been employed to characterize the profound genetic, socioeconomic, pharmacological, and clinical heterogeneity among SLE patients. Recent bulk and single-cell transcriptomic approaches in lupus have identified associations between inflammatory and interferon signatures with disease activity, ancestry, and autoantibody profiles³²⁻³⁴. An integrated multi-omic analysis of serum has recently hinted at dysregulated sphingolipid metabolism driving SLE activity³⁵, but otherwise, there have been few attempts to integrate findings across multiple profiles to establish systems-based mechanisms of disease characterizing SLE.

To take full advantage of the additional systems-level insights afforded by multi-omics, the present inventors isolated immune populations in bulk for a systems-focused analysis. The inventors were able to normalize signal differences due to cell type composition while maintaining a global perspective of the circulating immune environment.

The inventors applied this systems-based approach to two very different trials of abatacept in SLE. One is the investigator-initiated Clarification of Abatacept effects in SLE with integrated Biologic and Clinical approaches (ABC) trial, which aimed to assess the efficacy of abatacept in SLE patients without organ-threatening disease in a setting of minimal polypharmacy. This trial sought to address a major source of interference—background immunosuppression—in investigating biologic medications targeting components of the immune system, such as abatacept. By removing most background medication, this trial sought to reduce placebo response rates and enable detection of abatacept efficacy with a smaller trial size at a single site. To increase the power of the biological approach, the inventors also incorporated samples from the Abatacept and Cyclophosphamide Combination Therapy for Lupus Nephritis (ACCESS) trial, which provided a more suitable cohort for discovery. This trial specifically assessed the efficacy of abatacept as adjunctive therapy for lupus nephritis when co-administered with standard-of-care treatment low-dose cyclophosphamide followed by azathioprine. This was a large multi-site Immune Tolerance Network (ITN) trial with samples and data available through the ITN TrialShare network. These investigations had distinct entry and endpoint criteria that capture the heterogeneity of SLE across categories of age, race, sex, disease severity, and medication use.

ABC investigator-initiated clinical trial. The clarification of Abatacept effects in SLE with integrated Biologic and Clinical approaches (ABC, ClinicalTrials.gov #: NCT02270957) trial was a 6-month 1:1 randomized, double-blind, controlled phase IIb trial of abatacept and placebo with minimal polypharmacy in patients with active SLE, but no organ-threatening manifestations. This mirrored the design of the previous Biomarkers of Lupus Disease (BOLD) trial, which determined the safety of withdrawing immunosuppressive treatment from SLE patients with non-organ-threatening disease for the purpose of removing background polypharmacy to better study the lupus in the absence of immunomodulation18. After initial screening, patients could receive up to 320 mg total methylprednisolone by intramuscular injection as needed over the first month of the trial to suppress disease activity and manage symptoms. All background medications aside from some oral prednisone (<20 mg/day) were discontinued. Patients whose disease activity was sufficiently suppressed by steroids at one month were randomized 1:1 to receive weekly 125 mg abatacept or placebo by subcutaneous injection. This study was approved by the Oklahoma Medical Research Foundation (OMRF) institutional review board. All subjects provided written informed consent.

ABC Subjects. Eligible patients were men and women between 18 and 70 years or age that met at least four 1997 revised American College of Rheumatology (ACR) criteria for SLE. All had moderate to severe arthritis consistent with BILAG-2004 A or B with at least three swollen and tender joints. Patients with severe disease, including acute nephritis, CNS lupus, or any conditions requiring cyclophosphamide, biologics, or IV bolus steroids ≥500 mg were excluded. Overall, 76 patients were screened, of whom 66 met entry criteria and improved sufficiently with intramuscular steroids to comprise the intent-to-treat population for randomization.

ABC Endpoints and Assessments. The primary outcome measure was a 6-month assessment of the proportion of patients demonstrating sufficient improvement by the BILAG-based Combined Lupus Assessment (BICLA). BILAG refers to the British Isles Lupus Assessment Group Index. In this study, the 2004 iteration of this index was used⁴². The BICLA measure includes scores from BILAG-2004, the SLE Disease Activity Index (SLEDAI), and the Physician's Global Assessment (PGA)⁴³. BICLA response is defined as at least one BILAG-2004 letter grade improvement in each organ without an increase in SLEDAI and no more than a 10% increase in PGA. This SLEDAI measure is a hybrid index of the SLEDAI-2K44 and the SELENA-SLEDAI45. Secondary endpoints assessed in this trial included 6 month assessments of SLE Responder Index (SRI)-4, SLEDAI improvement, resolution of arthritis by SLEDAI, musculoskeletal improvement by BILAG, and achievement of low disease activity by a SLEDAI<2 or Lupus Low Disease Activity Score (LLDAS). Patients were classified as non-responders prior to or at month 6 if: (1) they dropped out of the study for any reason (lost to follow-up, adverse effects, etc.), (2) their disease flared, or (3) they initiated a new SLE medication outside of the allowed protocol treatment. Upon loss of response, patients could elect for any standard of care. After month 3, this could include open label abatacept.

Despite their wide use in clinical trials, BICLA and SRI indices are limited by the inherent pitfalls of their underlying instruments: the BILAG and SLEDAI, respectively. To address these, additional measures of flare severity employing visual analogue scales were used as exploratory endpoints. These were the Lupus Foundation of America-Rapid Evaluation of Activity in Lupus (LFA-REAL) and the SELENA SLEDAI Physician's Global Assessment (SSPGA). These measures have been shown to be reliable surrogates of more commonly used endpoints⁴⁶.

ACCESS Data, Samples, and Definition of Response. The results of the Abatacept and Cyclophosphamide Combination Therapy for Lupus Nephritis (ACCESS, ClinicalTrials.gov #: NCT00774852) trial have been previously reported¹⁹. As a participating trial, samples and data from ACCESS were available through the Immune Tolerance Network's (ITN) TrialShare program⁴⁷. PBMCs and serum samples from the ACCESS trial were obtained from the ITN biorepository by request to the ITN's TrialShare portal. De-identified demographic and clinical data on these samples was also obtained through the TrialShare portal.

ACCESS defined separate categories of complete response (CR), partial response (PR), and no response (NR) to abatacept at a 6-month time point. These were based on cutoffs for urine protein-to-creatinine ratio, serum creatinine improvement, and adherence to a prednisone taper. For this study, CR and NR patients in ACCESS were treated as abatacept responders and non-responders, respectively. Some partial responders demonstrated significant clinical improvement over their baseline scores, but did not meet the original cutoff criteria defined for CR or NR. These patients were treated as responders in this study.

Sample collection from the ABC trial. All samples used in this study were collected from screening visits in either the ABC or ACCESS trials. This is the first presenting visit, prior to any cessation of immunomodulating therapy while all patients have active disease. In the ABC trial, blood was collected at the first screening visit for isolation of peripheral blood mononuclear cells (PBMCs) and serum. Serum was aliquoted and stored at −80° C. for future analysis. PBMCs were isolated using Lymphocyte Separation Medium (Corning), and frozen at a concentration of 10 million/vial in freezing media (20% human serum/10% DMSO in RPMI) for storage in liquid nitrogen until future analysis.

Serum soluble mediator assessment. Serum levels of TGF-β1, APRIL, BMP2, MIP-1α, Eotaxin, IL-2Rα, GCSF, IFN-α, IFN-γ, IL-1Ra, IL-5, IL-7, IL-10, IL-17, IL-33, β-NGF, Resistin, E-selectin, TNF-α, VCAM-1, BAFF, MCP-1, MIP-1β, CD40L, FasL, ICAM, IFN-β, IL-1α, IL-4, IL-6, IL-8, IL-12p70, IL-21, LIF, PDGF-BB, SCF, Syndecan-1, TRAIL, VEGF, MIG, Fas, IL-2, IL-15, Leptin, TNF-RII, IP-10, IL-1β, IL-13, IL-23, TNF-RI, and CCL7 were assessed using custom xMAP Human Magnetic Luminex assays (R&D Systems, Inc.) according to manufacturer instructions. CCL7 was not measured in the ABC samples, but all other analytes listed were analyzed in serum samples from both samples. Data were analyzed on the Bio-Rad BioPlex200® array system (Bio-Rad Technologies), with a lower boundary of 50 beads per analyte per sample. The common 50 analytes shared between trials were used for downstream analysis.

Isolation of cell types of interest. Frozen PBMCs were thawed at 37° C. in RPMI supplemented with 4% FBS, 100 U/mL Penicillin, 100 U/mL Streptomycin, 2 mM glutamine, benzonase, and 3 μM actinomycin D. Cells were pelleted by centrifugation at 500 rcf for 10 minutes and then resuspended in autoMACS rinsing solution (Miltenyi biotec) supplemented with 5% MACS BSA stock solution and 3 μM actinomycin D. B cells were isolated from PBMCs using CD19 MicroBeads (Miltenyi Biotec) according to the manufacturer's protocol. Monocytes were isolated from the CD19-fraction from the previous step with CD14 MicroBeads (Miltenyi Biotec) according to the manufacturer protocol. T cells were isolated from the CD14-fraction from the previous step with the Pan T-Cell Biotin-AB Cocktail and Pan T-Cell MicroBead Cocktail (Miltenyi Biotec) according to the manufacturer's protocol. After isolation and counting of each cell type, at least 150,000 cells were resuspended in 50 μL of separation buffer and 50 μL of DNA/RNA Shield 2× Concentrate (Zymo Research). Samples were frozen at −80° C.

Cell type nucleic acid isolation. Nucleic acids were extracted from isolated cell fractions using the Quick-DNA/RNA Kit (Zymo Research). Frozen samples in DNA/RNA shield were thawed at room temperature, and 5 μL proteinase K and 10 μL PK Digestion Buffer (Zymo Research) were added and incubated at 55° C. for 30 minutes. Samples were transferred to Zymo-Spin IC-XM Columns (Zymo Research) and DNA and RNA were sequentially extracted according to the manufacturer's protocol. Final extractions were done with two elutions of 15 μL nuclease-free water for a total volume of 30 μL.

Nucleic acids were quantitated using Qubit DNA and RNA HS and BR Assays (Invitrogen), as needed. Genomic DNA was frozen at −20° C. for later analysis. RNA integrity was assessed using RNA 6000 Nano and Pico kits (Agilent) as needed on an Agilent 2100 Bioanalyzer to assess RNA Integrity Number (RIN). RINs for the samples had a median of 7.4, with the interquartile range falling between 6.6 and 8.2. Some RNA samples demonstrated signs of genomic DNA contamination and required additional cleanup. DNase I reaction buffer and DNase I (New England Biolabs) were added to RNA samples and incubated at 37° C. for 30 minutes. These samples were then purified with 1.8× volumes of Agencourt RNAClean XP beads (Beckman Coulter) according to protocol. RNA was eluted with 30 μL nuclease-free water and frozen at −80° C. for downstream analysis.

Cell type-specific RNA sequencing. Library preparation for RNA sequencing (RNA-seq) was carried out using the Advanta RNA-Seq XT NGS Library Prep Kit II (101-9959) (Standard Biotools). cDNA libraries were generated from 20 ng total RNA per sample, which were randomized across integrated fluidic circuits (IFCs) by cell type and abatacept response status. The Advanta RNA-Seq XT NGS kit generates dual-indexed, randomly primed cDNA libraries from the poly(A) siRNA contained within a total RNA sample. The Atlas IFC and Juno System™ (Standard Biotools) were used to automate poly(A) RNA capture by oligo-dT beads, washing, elution, fragmentation, and first strand cDNA synthesis using random primers. This was followed by second-strand cDNA synthesis with a template switch oligomer. Illumina adaptor and index barcodes were added to each library to enable sequencing demultiplexing. Individual sample libraries were isolated from the Atlas IFC and quantified for normalization via qPCR using the KAPA Library Quantification Kit for Illumina Platforms 480 (Roche) along with qPCR Primer Premix provided with the Advanta RNA-Seq XT NGS Library Prep Kit II. For each IFC run, normalization data was used to make 6 pools of barcoded libraries of similar quality and quantity. The pools were purified using Agencourt AMPure XP magnetic beads (Beckman Coulter) and further library enrichment was accomplished by another round of PCR using P5 and P7 primers. The product of this PCR was again purified using Agencourt AMPure XP magnetic beads and quantified using the KAPA Library Quantification Kit for Illumina Platforms that was optimized for the Roche LightCycler 480. A final library pool was generated that combined up to 48 uniquely indexed samples.

Sequencing was carried out by the OMRF Clinical Genomics Center on an Illumina NovaSeq 6000. Separate sets of “A” and “B” indices generated from individual Advanta kits were combined when possible to generate pools of up to 96 uniquely indexed samples. Pooled libraries were submitted for sequencing on individual lanes of an S4 flowcell to give an approximate depth of 50 million paired-end reads/library for cell type libraries.

Cell type-specific DNA methylation arrays. Frozen gDNA was thawed at room temperature and 100-500 ng was subjected to bisulfite conversion using the EZ-96 DNA Methylation Kit (Zymo Research). Conversion buffer was prepared immediately prior to reaction by adding provided 7.5 mL of nuclease-free water and 2.1 mL M-dilution buffer to the solid CT Conversion Reagent and vortexed for 10 minutes. After dilution in M-dilution buffer and nuclease-free water in deep well plates, samples were incubated in a thermal cycler for 16 cycles of 60 minutes at 50° C. followed by 30 seconds of 95° C. Samples were then held at 4° C. for at least 10 minutes and up to 20 hours. Samples were transferred to Silicon-A Binding Plates containing 400 μL of M-Binding Buffer before being centrifuged at 5000 rcf for 5 minutes. Converted DNA was washed with 400 μL of M-Wash Buffer, and plates were centrifuged on top of Collection Plates at 5000 rcf for 5 minutes. Collection Plates were emptied as needed in subsequent steps to prevent overflow. M-Desulphonation Buffer (200 μL) was added to each well and samples were incubated for 15 minutes at room temperature before being centrifuged at 5000 rcf for 5 minutes. Two more washes with 500 μL M-Wash Buffer were performed, with a 10 minute centrifugation on the final wash. Elution buffer (12 μL) was used to elute the contents of the Binding Plates into Elution Plates. After elution, 5 L of each sample was immediately delivered to the OMRF Clinical Genomics Center for methylation analysis using Infinium MethylationEPIC Arrays (Illumina).

Data preprocessing and quality control (QC). Serum soluble mediators. Median fluorescence intensity (MFI) for each analyte was interpolated from 5-parameter logistic nonlinear regression standard curves and used to generate an observed concentration (pg/mL). Out of range (OOR) negative values were interpreted as 0.001 pg/mL. OOR high values were handled in the following manner: A per plate maximum (P) was determined as the highest non-extrapolated value on a plate (including standards). Treating all extrapolations as actual values, high values were replaced using this formula with initial value V and final value F: If V=“OOR>” OR V>P, then return V=P+0.01, else return V.

RNA sequencing. Raw basecall (.bcl) files were converted to fastqs and demultiplexed using bcl2fastq (v2.20.0.422). Fastq files were further processed via a custom Nextflow pipeline48. Reads were trimmed via bbduk (39.01) to remove adapter sequences, poly(A) sequences, and rRNA sequences. Trimmed reads were pseudoaligned with salmon (1.3.0)⁴⁹ to the GRCh38 build (GENCODE 32)⁵⁰ of the human transcriptome for transcript quantification. Downstream analysis of quantitated counts was carried out in R (4.1.0)⁵¹ EdgeR (3.34.0)⁵² and limma (3.48.3)⁵³ were used to normalize transcript count data per sample, accounting for library size, cell type, and responder status in the design.

DNA methylation arrays. Raw data, received as .idat files, were preprocessed in R using the minfi (1.38.0)⁵⁴ package. Data was normalized with the preprocessFunNorm function. QC was performed by calculating detection p-values for each sample across all probes with the detectionP function. Samples with mean p-values>0.01 were removed from further analysis, and probes with any p-values>0.01 were also removed from further analysis. Normalized values were returned as the proportion of total sites methylated (beta values).

Machine Learning. A bootstrapped resampling approach was employed to develop and approximate performance of machine learning models. After QC and normalization to beta values, input DNA methylation data was split into 70% training and 30% testing data 40 times. Each of these resampled splits would undergo development for generalized linear models (GLMs) using the glmnet (4.1-2)⁵⁵ package in R. The cv.glmnet function was applied to training splits of the data with default parameters to fit logistic regression classifiers (responders vs. non-responders) with Least Absolute Shrinkage and Selection Operator (LASSO)-based regularization. Tuning of the lambda parameter was done with 10-fold cross-validation on the training sets. The best model was used to predict the classification on the testing split. Performance as area under the curve (AUC) of the receiver operator characteristic (ROC) and feature usage statistics of the 40 bootstrapped models were collected for reporting. Data for plotting ROC curves was generated using the ROCR (1.0-11)⁵⁶ package. “Averaged” ROC curve points were generated by taking the mean sensitivity and specificity values generated by ROCR across the 40 bootstrapped models.

CpG annotation. Annotation of CpGs was done through the missMethyl (1.26.1)⁵⁷ package, which provides chromosomal locations of probe sites, near variants, regional annotations, and nearby genes for Infinium MethylationEPIC array probes. The local genomic impact of methylation at critical sites identified by machine learning was studied similarly to genetic linkage between nearby genomic sites. This methylation linkage was calculated as the coefficient of determination (R2) between the methylation beta values at a CpG of interest and the beta values of nearby CpGs. In this way, a higher linkage score (R2) indicates a greater association in the methylation of proximal CpGs assayed.

Interomic analysis. Interomic analysis was performed by correlating methylation at CpG sites of interest with transcriptomic data. To do this, normalized methylation as beta values were transformed to M-values by a logit transformation (Equation 1). Linear models were then built with the limma package correlating methylation M-values at each CpG site with normalized cell type-specific transcription data. P-values in this approach were corrected for multiple comparisons by Benjamini-Hochberg adjustment. Effect sizes reported by these tests indicated the slope of the linear model fit to these relationships.

$$M = {log}_{2} ( \frac{β}{1 - β} ) . \qquad Equation 1$$

Differential methylation analysis. Differential methylation analysis of probe sites was also performed using the limma package. Normalized methylation M-values, as calculated above, were used for cell type-specific analysis. To account for confounding technical batch effects, surrogate variable analysis (SVA) was employed to identify 2 technical covariates of the data not captured by cell type or abatacept response status. These technical covariates were incorporated into the final linear model of differential methylation. Filtering of these data was performed using the CpG annotation noted above, with regional annotations of “TSS200” and “TSS1500” indicating probes falling within 200 bp and 200-1500 bp, respectively, of transcription start sites (TSSs).

Ingenuity Pathway Analysis. Systems-level analysis was undertaken with QIAGEN Ingenuity® Pathway Analysis (IPA). Transcript correlation data was used as input with a significance cutoff of adjusted p-value<0.05 and effect size cutoffs identified independently for each CpG marker. These were absolute slope values of 1.5 for cg04612171, 0.5 for cg16733676, 0.2 for cg02901522 and 1 for cg11986743. Differential methylation of transcription start sites was used as input with a significance cutoff of adjusted p-values<0.05 and effect size cutoffs of an M-value difference of at least 0.3, which corresponds to the smallest possible beta value difference of 0.05. Genes with multiple significant CpGs near their TSS were resolved using the highest absolute M-value difference.

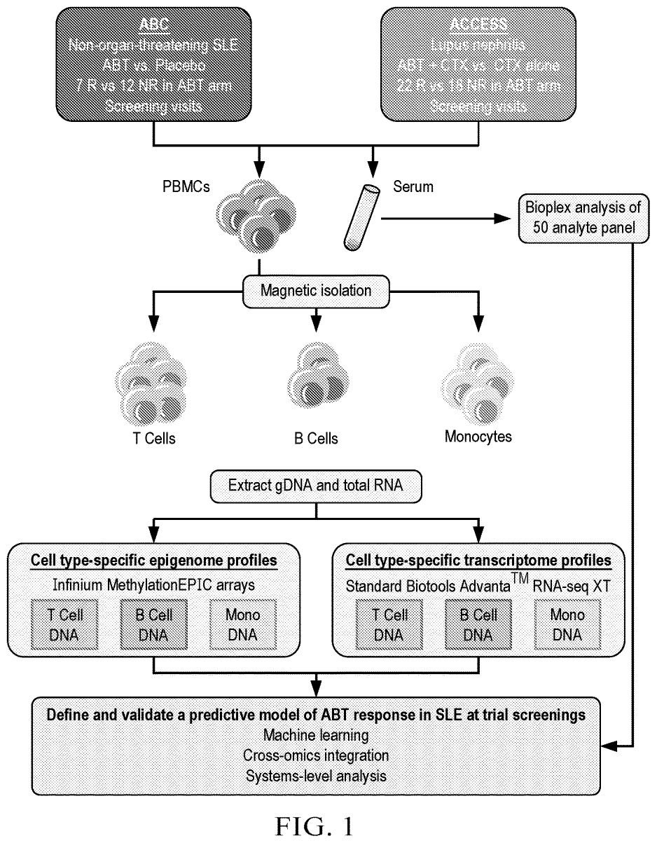

A systems biology approach to assess drug response in two trials of abatacept in lupus. The primary goal of this study was to develop a predictive model of abatacept response in lupus using data from the screening visits of these clinical trials. A systems biology approach was chosen to dissect contributions from underrepresented circulating immune populations (FIG. 1). This allowed for less frequent cell types (i.e. B cell subsets) to be analyzed with the same depth as more abundant cell types, effectively normalizing heterogeneity due to cell type abundances. PBMCs and serum were collected from screening visits of identified responders and non-responders in the ABC and ACCESS trials. Soluble mediators were assessed in serum by a 50-analyte panel by Bio-Rad Bioplex ANA2200 assay. PBMCs were fractionated by successive antibody-conjugated magnetic bead-based isolations to obtain major immune populations of B cells, monocytes, and T cells. Genomic DNA and total RNA were sequentially extracted from these bulk populations and used to generate parallel transcriptome and epigenome profiles. These molecular profiles were then used for an integrated analysis to develop a model of abatacept response incorporating findings across multiple cell types and omics layers.

Machine learning identified a four-marker methylation signature distinguishing abatacept responders and non-responders at screening. The cohort of ACCESS screening samples was considerably larger and more balanced than the ABC samples. As it was conducted in the setting of lupus nephritis, this trial was also predicted to have greater measurable biological differences between responders and non-responders than in the ABC trial, which examined SLE patients with active, but less severe disease. The inventors implemented a machine learning approach to first develop a model of abatacept response in ACCESS trial patients from DNA methylation. After final QC, 865,859 CpG sites across 119 total samples from ACCESS were used as input for machine learning model development. A bootstrap approach was utilized to generate 40 random splits of the samples into 70/30 training/testing portions for development and testing of generalized linear models (GLMs) as described in methods. These 40 initial models robustly separated responder samples from non-responder samples, with an average AUC of the ROC for test samples of 0.982 and an average test error of 6.36%. These models used between 15 and 47 features total, with a median of 32 CpG methylation values used. The inventors examined the frequency of marker usage across bootstrapped divisions, noting that the use of a set of 13 CpGs was shared among more than half of the models generated. These 13 CpGs were used as the sole input to construct parsimonious models across all ACCESS samples, using the same strategy of 40 permutations of training-testing splits, which resulted in highly accurate models that produced an average test AUC>0.999 and an average test error of 0.929%. Finally, the inventors evaluated these 13-feature models on the ABC dataset, which yielded an average AUC of 0.659 and an average test error of 39.4%. While not very high performing, the model showed some discriminative ability despite the vastly different clinical settings of both trials.

While their predictive power was weaker, that these 13 CpGs identified and modeled in a lupus nephritis trial were able to distinguish abatacept response in a non-organ-threatening setting suggested an underlying importance in SLE pathology. Next, the inventors examined how responders and non-responders differed in the methylation status at these sites independently in ABC and ACCESS. It was noted that while the differential methylation between responders and non-responders had overall far less power in the ABC trial, there were several markers that maintained common trends between both trials. These were cg02901522, cg16733676, cg11986743, cg04612171, cg17988158, and cg12421087. Other CpG sites had inconsistent patterns and distributions of methylation, which may be due to the different populations of lupus patients studied. However, the presence of common patterns of methylation across both trials suggested that these markers would perform well in a model developed on patient samples from both trials.

The inventors applied a machine learning approach to a combined cohort of ABC and ACCESS. Bootstrapped models separated responder samples from non-responder samples robustly, with an average AUC of 0.976 and an average error of 6.78%. The models used between 20 and 67 CpG features, with a median of 38.5 markers used. Specific markers were consistently used across these bootstrapped divisions, with the top four features being used in at least 35/40 development models. These four CpGs had also shown the most consistent pattern of methylation between responders and non-responders across both the ABC and the ACCESS trial, confirming the evaluation of the previous signature. Eliminating these four CpGs and attempting to model abatacept response yielded a model which performed slightly less well, with a mean AUC 0.955 and an average error of 8.07%. These models used more CpGs (median=50), and had more variable performance (AUC SD=0.0547). There was also more variety in the CpGs utilized, with the most frequently used marker used in 34/40 of models. These data demonstrate the importance of methylation at these four CpGs in predicting response to abatacept treatment.

Since abatacept is thought to primarily target co-stimulation of T cells, the top four features were used to construct parsimonious models on T cells alone. T cell samples were used as input to the same strategy of 40 permutations of training-testing splits using only the top four most commonly used features in model development. This yielded very robust models, with an average testing AUC of 0.935 and an average testing error of 16.0%. Applying these same models to B cell and monocyte samples yielded similarly reliable classification, with average AUCs of 0.964 and 0.949, respectively. Average testing errors were 13.6% on B cells and 20.7% on monocytes. Confusion matrices of the performance of the models on monocyte samples reveals that the relatively higher error rates stem from a high proportion of false positives (Table 3.5). Even though the models were trained on T cells, they maintained overall predictive performance when assayed on B cells and high negative predictive value when assayed on monocytes. The parsimonious models constructed in this approach identify a reliable predictive signature across PBMC compartments from these four selected markers.

The ABC and ACCESS trials studied the efficacy of abatacept in different clinical contexts regarding lupus severity and end-organ involvement. To further confirm the performance of the parsimonious models in each of these contexts using the present invention, the models developed on all T cells were assessed on samples from the ACCESS and ABC trials independently. The models distinguished response in ACCESS T cells very consistently, with an average AUC of 0.974 and an average error rate of 10.2%. Model performance on ABC T cells had a smaller average AUC of 0.868 and an average error rate of 24.1%. Additionally, the parsimonious models trained on the ABC T cell samples maintained performance when validated on T cell samples from the ACCESS trial, and vice-versa. While the model consistently performs better on ACCESS samples than on ABC samples, possibly due to the overrepresentation of these samples, the model was robust despite the very different clinical contexts of the samples examined. This is supported by the similar patterns of methylation between studies.

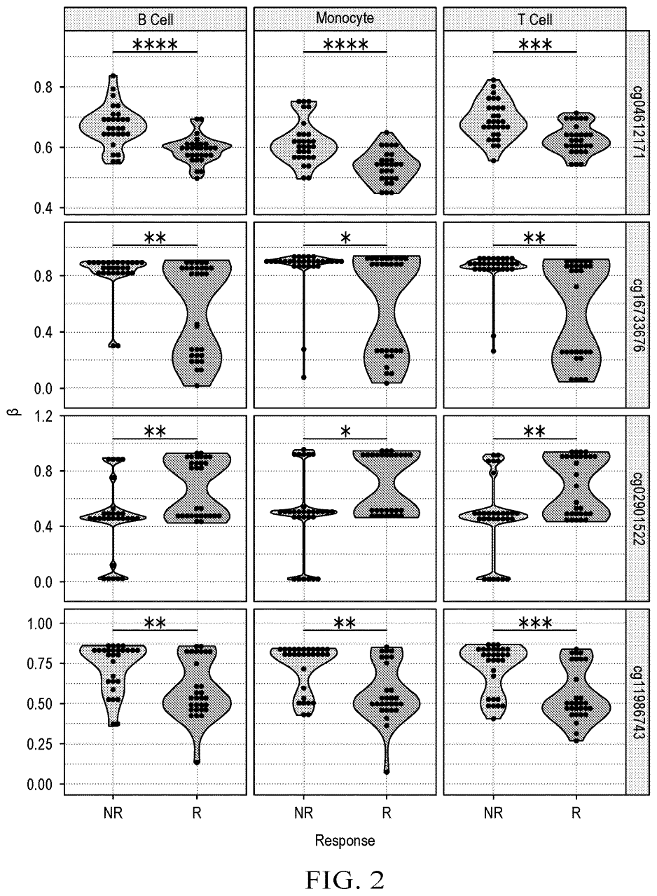

Identified methylation markers represent both epigenetic and genetic influence at predicted genomic regulatory regions. The inventors examined the methylation status of the top four features selected in model development across cell types and response status in the dataset (FIG. 2). All four markers exhibited a statistically significant difference in proportion of sites methylated (beta values) in all isolated PBMC fractions. The top performing marker, cg04612171, had a continuous distribution of methylation beta values ranging from 0.45 to 0.85, as expected of a single CpG assayed in a single cell type across a population. However, the other three markers, cg16733676, cg02901522, and cg11986743, had pronounced bimodal or trimodal distributions of methylation beta values centered near 0, 0.5, and 1. These three markers all correspond to CpG sites which include highly common G>A,C SNPs immediately subsequent to the assayed meC position (Table 1). These indicate a potential genetic mechanism by which DNA methylation at these loci is controlled.

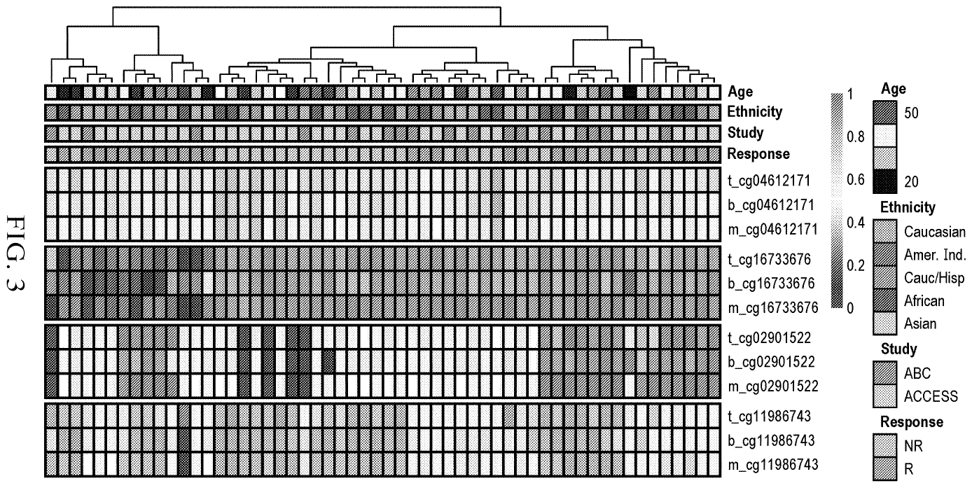

Using only methylation at these three variant-associated CpGs to model response in T cells was successful, with an average testing AUC of 0.893 and an average testing error of 23.4%. Methylation of each marker in each cell type was compared across individuals. Hierarchical clustering on these data largely separated patients into a mostly-responder cluster, a mostly non-responder cluster, and a mixed cluster (FIG. 3). The methylation status of the four markers was highly coordinated across cell types. This was expected at the three variant-associated CpG loci identified herein, given the genetic control of methylation, but also held for the methylation at probe site cg04612171. The four methylation sites which form the parsimonious models of response represent a differential biomarker signature that distinguishes abatacept responders from non-responders.

TABLE 1
Genomic annotation of top four CpGs predictive of abatacept response

| | | 5meC | Proximal | SNP | Minor Allele | Proximal | GENCODE |
|---|---|---|---|---|---|---|---|
| CpG ID | Chromosome | Position | SNP | Position | Frequency | Gene | Annotation |
| cg04612171 | 6 | 107814572 | rs573616248 | 0 | 0.0002 | SOBP | Body |
| cg16733676 | 1 | 108734992 | rs521545 | 1 | 0.2841 | SLC25A24 | Body |
| cg02901522 | 8 | 129082086 | rs2250962 | 1 | 0.3021 | PVT1 | Body |
| cg11986743 | 18 | 29205358 | rs41447647 | 1 | 0.1543 | B4GALT6 | 3′UTR |

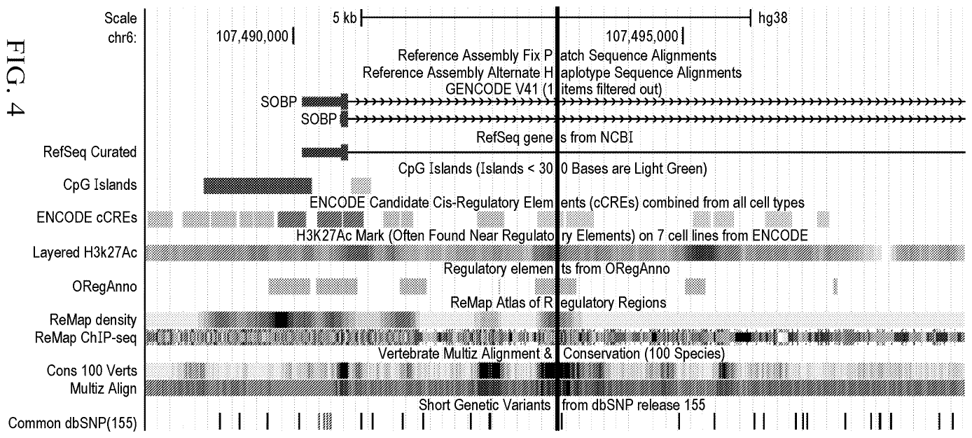

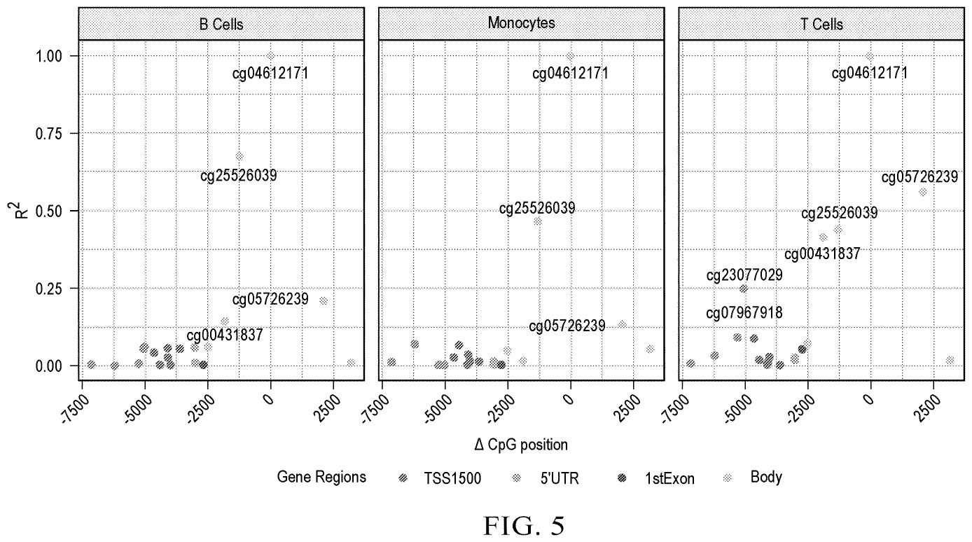

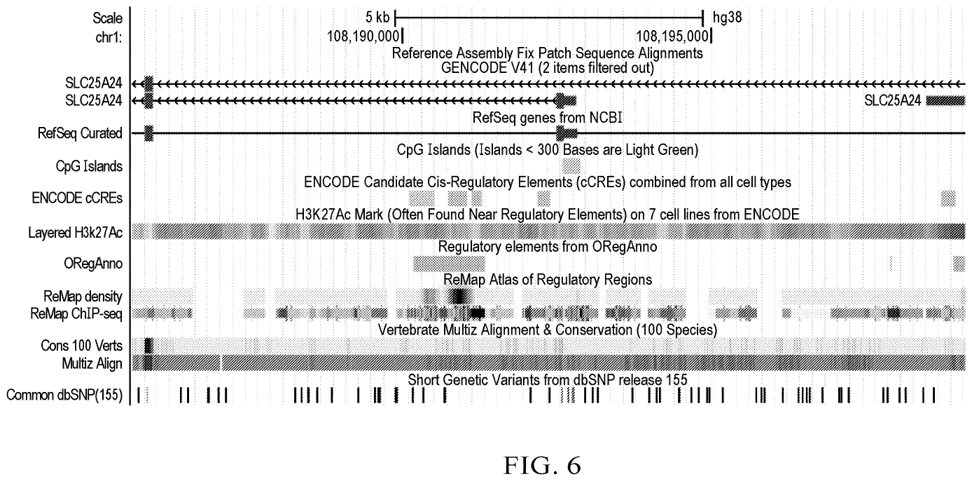

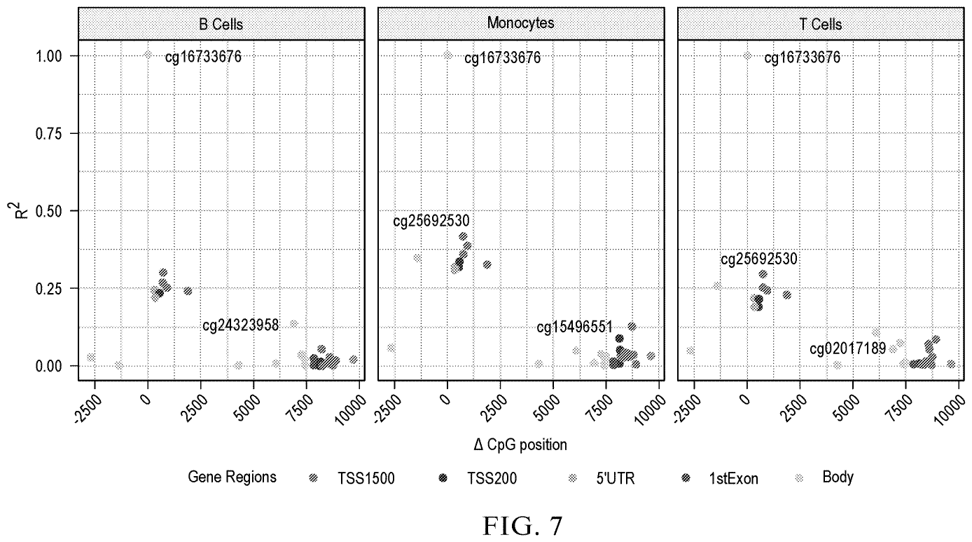

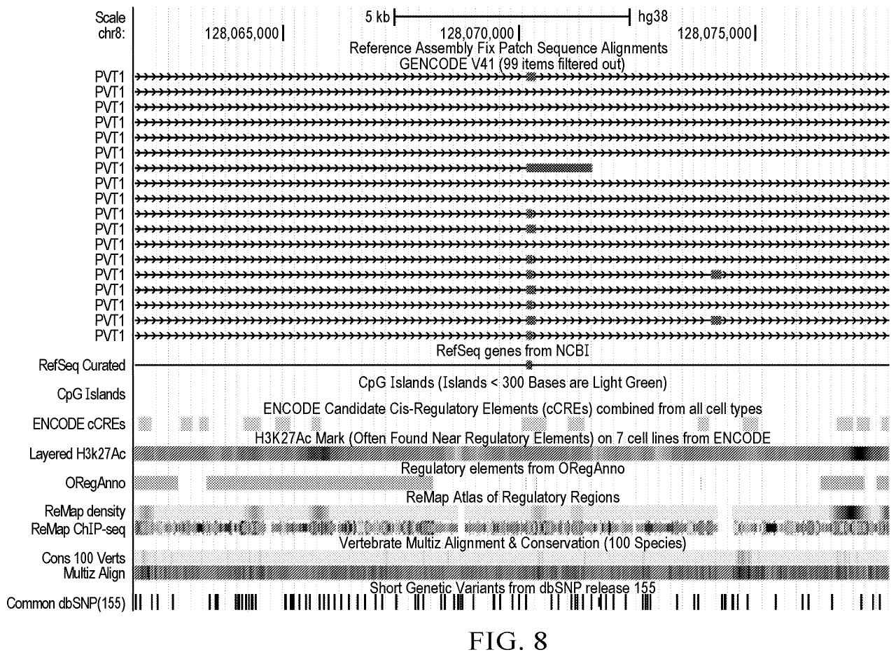

The inventors examined the methylation at these four sites for annotated activity and inspected their impact on their respective local methylation landscapes. The cg04612171 CpG site falls within a putative cis-regulatory element in the first intron of the Sine Oculis Binding Protein Homolog (SOBP) gene (FIG. 4). This region is also dense with H3K27 acetylation and reported ChIP-seq transcription factor binding sites. Methylation at this locus appears to be linked to methylation at CpGs further downstream in the gene body of SOBP, primarily in T cells (FIG. 5). Marker cg16733676 corresponds to an upstream shore of the CpG island in the gene body of Solute Carrier Family 25 Member 24 (SLC25A42) (FIG. 6). While this site appears to not reside within any known or approximate regulatory region, it does have a strong apparent linkage with methylation status near the TSS of SLC25A24 (FIG. 7). This relationship is maintained across cell types but is most prominent in T cells and monocytes. Correspondingly, normalized counts from RNA-seq of SLC25A25 across cell types similarly correlates with methylation at this locus in T cells and monocytes (FIG. 8). As previously noted, the CpG at this site contains a highly common SNP which appears to correspond to bimodal levels of methylation across cell types. The association of this SNP both with methylation at the proximal TSS and also with the transcript level of the target gene implies function as a cis-acting methylation quantitative trait locus (cis-meQTL). Presence of the minor allele impairs methylation at this site, which is linked to decreased methylation at the TSS, resulting in lower levels of the SLC25A24 transcript.

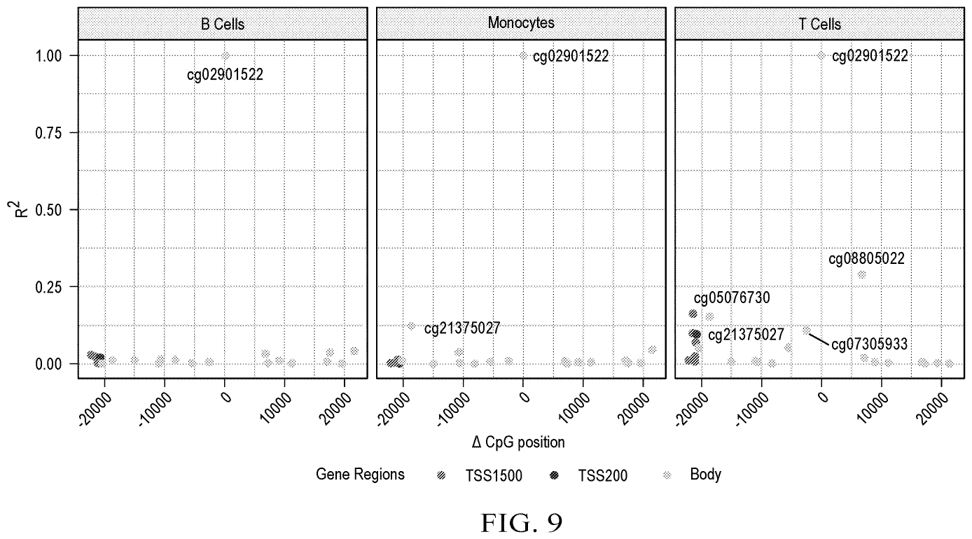

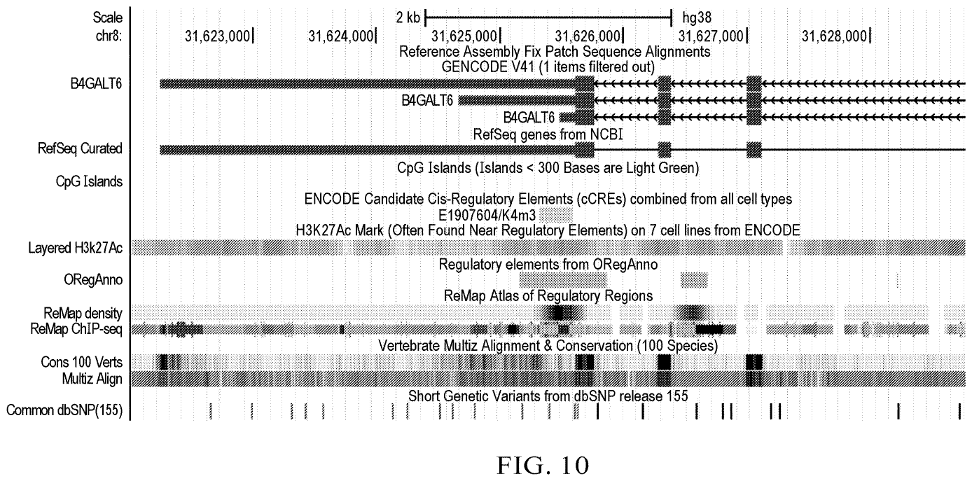

The CpG corresponding to the cg02901522 probe is located on chromosome 8 within an intronic region of the Plasmacytoma Variant Translocation 1 (PVT1) transcript (FIG. 9). This is a long non-coding RNA (lncRNA) transcript with multiple isoforms and is located downstream of MYC, a transcription factor governing global cellular transcription. This site is located in another predicted regulatory site in an intron preceding the fifth annotated exon. Methylation at this SNP-associated CpG appears to be linked to a nearby CpG containing another common variant in T cells (FIG. 10). This represents a T cell-specific methylation linkage at this region.

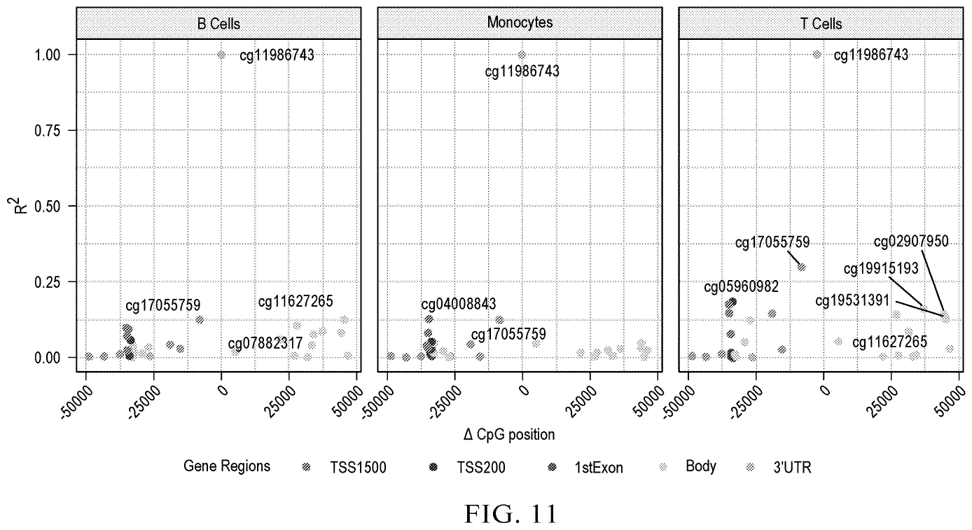

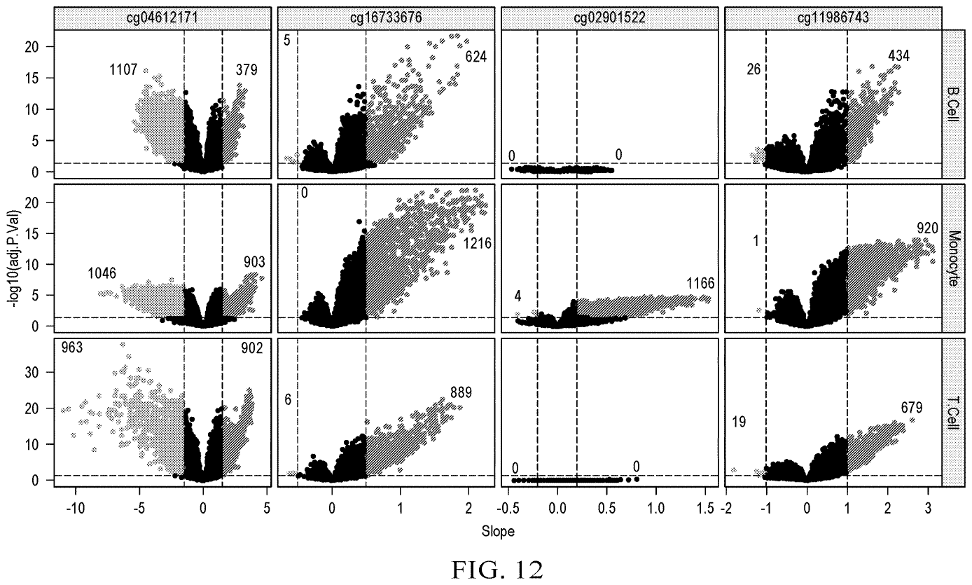

Finally, cg11986743 represents a CpG in another putative regulatory site located near the 5′ UTR of B4GALT6 (FIG. 11). It does not appear to be linked to proximal methylation or with transcription of the immediate gene (FIG. 12). The methylation signature was identified as predictive of abatacept response in lupus patients contains four sites with predicted regulatory activity. Evidence at one site predicts function as an unannotated meQTL but the impacts of methylation at the other sites remain largely uncharacterized.

Epigenetic response signature linked to cell type-specific gene expression implicating abatacept-targeted pathways. Using parallel cell type-specific RNA-seq data, whether methylation at the predictive loci correlated with global gene expression in cell types was determined. To do so, the inventors correlated methylation at each of the four CpGs used in the parsimonious abatacept response models to gene expression profiles completed on a subset of samples. Significant gene expression was both positively and negatively correlated with methylation at the SOBP locus, while significant correlations of transcripts to the methylation at the three variant-CpGs were mostly positive. Additionally, distinct gene expression associations were observed within each cell type with variant and non-variant CpG methylation. The CpG at the PVT1 locus demonstrated significant correlations in monocytes, while methylation at the other three loci had significant transcript impacts across all assayed cell populations.

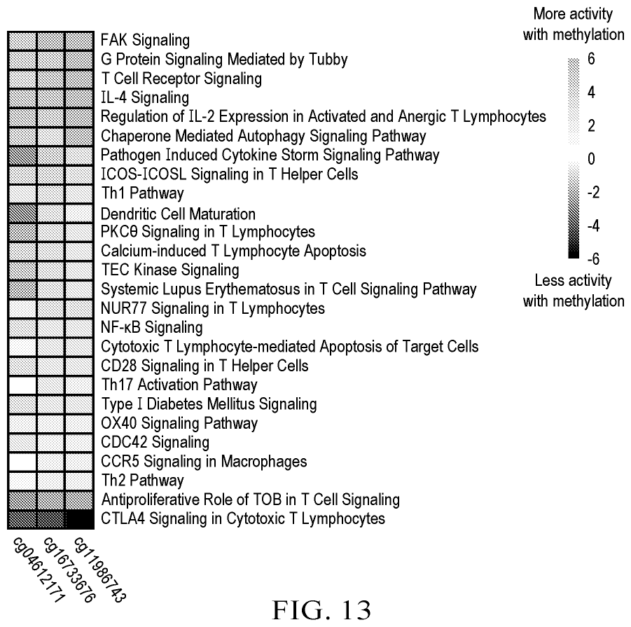

In T cells, for cg04612171 methylation the top positively correlated genes included TCR variant genes, XCL2, FASLG, and VCAM1, while the top negatively correlated genes included myeloid-associated genes LYZ, CD300E, SPI1, and CSF3R. Methylation in the two variant-linked CpGs correlated most strongly with markers of T cell activation including ICOS, CD28, and CD6, as well as the regulatory markers LEF1 and PLXNA4 (FIG. 13). B cells had major positive associations between cg04612171 methylation in Ig light chain genes, while the top negative associations included CD36, PTGER2, and ITGA6. Variant-associated methylation was linked most strongly to markers of B cell lineage, MS4A1, CD79A, BLK, BANK1, PAX5, and CD19. FOLR3, WNT5A, and CCL7 were among the most positive associations with cg04612171 methylation in monocytes, and lymphoid markers LEF1, PAX5, and ZAP70 were among the most negative correlations. Methylation in all 3 variant CpGs had the strongest positive correlations with myeloid lineage and activation markers such as CD14, S100A8/9, CD68, VCAN, and TREM1. The number of strong transcriptomic correlations observed across cell types with methylation at these markers provides a signature associated with functional differences in major immune subpopulations.

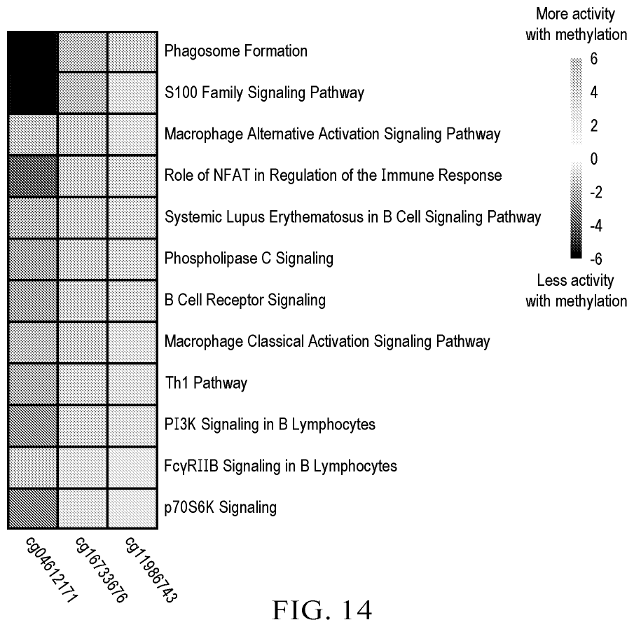

Ingenuity pathway analysis was implemented to identify canonical pathways and associated activity in the methylation-correlated transcripts. Cutoffs for significance and slope of the linear regression were applied. In T cells, the most significant and strongest canonical pathways associated with methylation at three loci were “CTLA4 regulation of CD8+ T cell-mediated cytotoxicity”, “T Cell Receptor Signaling” and “Regulation of IL-2 production by T cells”, indicating a major association with T cell signaling and its regulation (FIG. 14). In addition to enrichment analysis, IPA also provides a prediction of activity in the associated pathway, an activity z-score, by comparing the provided correlation direction with the expected gene change associated with activity in the pathway. The negative z-score less than-2 indicates that activity in the CTLA4 pathway is predicted to be negatively correlated with methylation in three marker CpGs. Likewise, T cell receptor activity and IL-2 production are predicted to be positively associated with methylation at the same loci. This indicates a critical link between the modeled loci and the regulation of the costimulatory signal to T cell activation which is targeted by abatacept. More methylation at the CpGs of interest is positively correlated with T cell activation via the T cell receptor, which is predictive of non-response to abatacept.

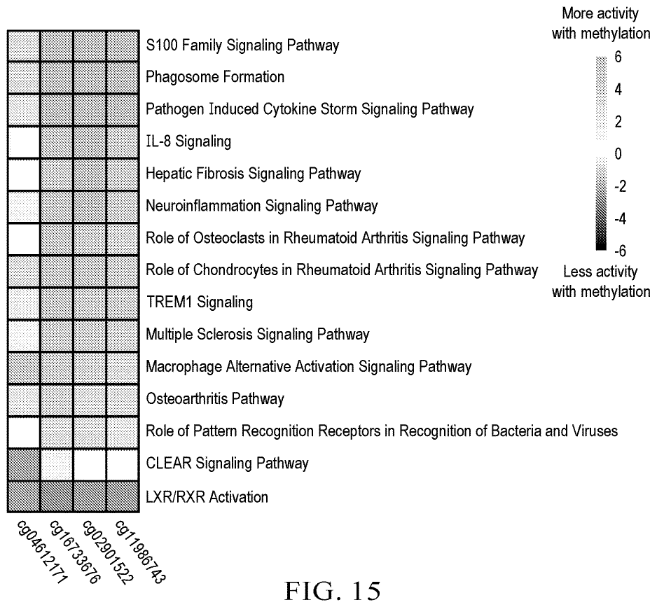

FIG. 15. Ingenuity Canonical Pathway Analysis of monocyte transcriptome correlations with methylation at CpG sites of interest. Summary of most significant results comparing Canonical Pathway Analysis of by QIAGEN's Ingenuity® Pathway Analysis (IPA). The most significantly correlated monocyte transcripts with methylation at each CpG of interest were input into IPA with previously noted cutoffs. Rows represent the most significant results across all analyses, and columns represent the correlations with each CpG site noted at the bottom. Color indicates the activation z-score, which represents a predicted change in pathway activity correlating with methylation.

Similar analysis carried out in the other isolated cell types also links this epigenetic signature to pathogenic immune activation. While B cell receptor (BCR) activity is significantly enriched in genes correlated with CpG methylation, the direction of the activity correlation is inconsistent between markers. BCR activity is negatively correlated with methylation at the SOBP gene body site, and it is positively correlated with methylation at two variant-associated CpG sites. Monocyte transcripts correlated with epigenetics are enriched in “Phagosome formation”, “Cytokine storm” and “RXR activation” canonical pathways (FIG. 16). Further, there is consistent positive correlation of the proinflammatory phagocytosis and cytokine pathways with methylation of model CpGs, as well as consistent negative correlation with anti-inflammatory retinoic acid pathways. Abatacept inhibits T cell activity via disruption of co-stimulation, but the correlations of canonical pathways with this predictive methylation signature indicate parallel immune activation of monocytes and B cells playing a role in disease response.

To examine whether methylation at these loci were linked to any soluble biomarkers, the inventors also examined serum concentrations of a panel of 50 markers which were commonly measured in both trials. Levels of resistin measured at this same visit were positively correlated with methylation at the site in the SOBP gene body. This correlation was most pronounced in B cells and T cells, but monocytes also showed weak correlations. Resistin is an adipokine which has been previously linked to renal dysfunction in lupus nephritis compared to healthy individuals. Additionally, other proinflammatory cytokines, notably IL-6 and IL-17, showed trending negative correlations with methylation at several markers which didn't meet significance due to sample size. The pro-inflammatory role of resistin may be linked to methylation in PBMCs.

In conclusion, it was found that cell-specific methylation signatures are predictive of the responsiveness to a drug that targets the co-stimulatory pathway. The genes impacted by the methylation signature can be directly mapped to both upregulated expression and downregulated expression of the predicted drug-targeted genes through pathways in the cell. The methylation signature predicts response very well in two clinical trials with very disparate patient characteristics undergoing treatment and different impacts of background medications. Finally, a systems immunology approach leveraging multi-omic data and machine learning can be a powerful approach to better understanding underlying mechanisms and can be used for precision medicine.

A person of skill in the art would readily recognize that steps of various above-described methods can be performed by programmed computers. Herein, some embodiments are also intended to cover program storage devices, e.g., digital data storage media, which are machine or computer-readable and encode machine-executable or computer-executable programs of instructions, wherein said instructions perform some or all of the steps of said above-described methods. The program storage devices may be, e.g., digital memories, magnetic storage media such as magnetic disks and magnetic tapes, hard drives, or optically readable digital data storage media. The embodiments are also intended to cover computers programmed to perform said steps of the above-described methods.

The functions of the various elements shown in the figures, including any functional blocks labeled as “modules”, may be provided through the use of dedicated hardware as well as hardware capable of executing software in association with the appropriate software. When provided by a processor, the functions may be provided by a single dedicated processor, by a single shared processor, or by a plurality of individual processors, some of which may be shared. Moreover, explicit use of the term “module” should not be construed to refer exclusively to hardware capable of executing software, and may implicitly include, without limitation, digital signal processor (DSP) hardware, network processor, application-specific integrated circuit (ASIC), field-programmable gate array (FPGA), read-only memory (ROM) for storing software, random access memory (RAM), and nonvolatile storage. Other hardware, conventional and/or custom, may also be included.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

Additionally, the section headings herein are provided for consistency with the suggestions under 37 CFR 1.77 or otherwise to provide organizational cues. These headings shall not limit or characterize the invention(s) set out in any claims that may issue from this disclosure. Specifically and by way of example, although the headings refer to a “Field of Invention,” such claims should not be limited by the language under this heading to describe the so-called technical field. Further, a description of technology in the “Background of the Invention” section is not to be construed as an admission that technology is prior art to any invention(s) in this disclosure. Neither is the “Summary” to be considered a characterization of the invention(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure, but should not be constrained by the headings set forth herein.

For each of the claims, each dependent claim can depend both from the independent claim and from each of the prior dependent claims for each and every claim so long as the prior claim provides a proper antecedent basis for a claim term or element.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit, and scope of the invention. All such similar substitutes and modifications apparent to those skilled in the art are deemed to be within the spirit, scope, and concept of the invention as defined by the appended claims.

### Embodiments

Embodiment 1. A method to select lupus patients that will be responsive to a treatment with an inhibitor of CD80/86 immune cell costimulation, comprising:
determining whether a patient has a genetic predisposition to being responsive to the treatment with the inhibitor of CD80/86 immune cell costimulation by:
obtaining or having obtained a whole blood sample from the patient with lupus;
separating T cells, B cells, and monocytes;
performing or having performed a methylation assay on a genome of at least one of the T cells, B cells, or monocytes to detect hypomethylation at one or more CpG biomarkers selected from;
Cg04612171;
Cg16733676;
Cg02901522; or
Cg11986743;
determining if the patient has hypomethylation at the one or more CpG biomarkers when compared to a subject that does not have lupus in the at least one of the T cells, B cells, and monocytes, and
administering the inhibitor of CD80/86 immune cell costimulation if the patient has hypomethylation of the one or more of the CpG biomarkers in the least one of the T cells, B cells, and monocytes; or
discontinuing treatment with the inhibitor of CD80/86 immune cell costimulation if the patient does not have hypomethylation of the one or more of the CpG biomarkers in the least one of the T cells, B cells, and monocytes.
Embodiment 2. The method of embodiment 1, wherein the inhibitor of CD80/86 immune cell costimulation is Abatacept or Belatacept.
Embodiment 3. The method of embodiments 1 or 2, wherein the hypomethylation is detected for any 2, 3 or 4 of Cg04612171 and Cg16733676; Cg02901522; and Cg11986743.
Embodiment 4. The method of any one of embodiments 1 to 3, wherein the hypomethylation is detected for any 2 or 3 of the T cells, B cells, and monocytes.
Embodiment 5. The method of any one of embodiments 1 to 4, wherein the hypomethylation is detected for any 2 or 3 of the T cells, B cells, and monocytes and is detected for any 2, 3 or 4 of Cg04612171 and Cg16733676; Cg02901522; and Cg11986743.
Embodiment 6. The method of any one of embodiments 1 to 5, wherein the hypomethylation is detected in T cells and there is an increase in T cell receptor (TCR) signaling genes, a decrease in T cell proliferation, or both.
Embodiment 7. The method any one of embodiments 1 to 6, wherein the hypomethylation is detected in monocytes and there is an increase in expression of genes in retinoic acid pathway genes.
Embodiment 8. The method any one of embodiments 1 to 7, wherein the hypomethylation is detected in B cells and there is a decrease in B cell receptor (BCR) activity, Fc gamma receptor IIB (FCγRIIB) activity, or both.
Embodiment 9. The method of any one of embodiments 1 to 8, wherein the hypomethylation is detected by bisulfite sequencing, high-performance liquid chromatography-ultraviolet (HPLC-UV), liquid chromatography coupled with tandem mass spectrometry (LC-MS/MS), enzyme-linked immunosorbent assay (ELISA), long interspersed nuclear elements (LINE-1) and Pyrosequencing, amplification fragment length polymorphism (AFLP), restriction fragment length polymorphism (RFLP), luminometric methylation assay, bead-chip, bead-chip array, microarray, methyl-sensitive cut counting, or luminometric methylation.
Embodiment 10. The method any one of embodiments 1 to 9, wherein the hypomethylation of Cg04612171 and Cg16733676; Cg02901522; and Cg11986743, affects expression of Sine Oculis Binding Protein Homolog (SOBP), Solute Carrier Family 25 Member 24 (SLC25A24), Plasmacytoma Variant Translocation 1 (PVT1), or Beta-1,4-Galactosyltransferase 6 (B4GALT6), respectively.
Embodiment 11. The method of any one of embodiments 1 to 10, wherein the lupus is systemic lupus erythematosus, Cutaneous lupus erythematosus, drug-induced lupus, or neonatal lupus.
Embodiment 12. The method any one of embodiments 1 to 11, further comprising the step of monitoring a methylation status of the one or more CpG biomarkers at one or more points in time and based on a change in methylation discontinuing or restarting treatment with the inhibitor of CD80/86 immune cell costimulation.
Embodiment 13. The method of any one of embodiments 1 to 12, further comprising the step of monitoring a methylation status of the one or more CpG biomarkers at one or more points in time of a patient undergoing treatment with the inhibitor of CD80/86 immune cell costimulation and based on a change in methylation selecting an alternative treatment.
Embodiment 14. A method of treating lupus patients that will be responsive for a treatment with an inhibitor of CD80/86 immune cell costimulation, comprising:
determining whether a patient has a genetic predisposition to being responsive to the treatment with the inhibitor of CD80/86 immune cell costimulation:
obtaining or having obtained a whole blood sample from the patient with lupus;
separating T cells, B cells, and monocytes;
performing or having performed a methylation assay on a genome of at least one of the T cells, B cells, or monocytes to detect methylation at one or more CpG biomarkers selected from;
Cg04612171;
Cg16733676;
Cg02901522; or
Cg11986743;
determining if the lupus patient has hypomethylation at the one or more CpG biomarkers when compared to a subject that does not have lupus in the at least one of the T cells, B cells, and monocytes, wherein the lupus is systemic lupus erythematosus, Cutaneous lupus erythematosus, drug-induced lupus, or neonatal lupus; and
administering the inhibitor of CD80/86 immune cell costimulation if the patient has hypomethylation of the one or more of the CpG biomarkers in the least one of the T cells, B cells, and monocytes; or
discontinuing treatment with the inhibitor of CD80/86 immune cell costimulation if the patient does not have hypomethylation of the one or more of the CpG biomarkers in the least one of the T cells, B cells, and monocytes and administering the patient with at least one of NSAIDs, corticosteroids, immunosuppressants, hydroxychloroquine, or methotrexate.
Embodiment 15. The method of embodiment 14, wherein the inhibitor of CD80/86 immune cell costimulation is Abatacept or Belatacept.
Embodiment 16. The method of embodiments 14 or 15, wherein the hypomethylation is detected for any 2, 3 or 4 of Cg04612171 and Cg16733676; Cg02901522; and Cg11986743.
Embodiment 17. The method of embodiments 14 or 16, wherein the hypomethylation is detected for any 2 or 3 of the T cells, B cells, and monocytes.
Embodiment 18. The method of embodiments 14 or 17, wherein the hypomethylation is detected for any 2 or 3 of the T cells, B cells, and monocytes and is detected for any 2, 3 or 4 of Cg04612171 and Cg16733676; Cg02901522; and Cg11986743.
Embodiment 19. The method of embodiments 14 or 18, wherein the hypomethylation is detected by bisulfite sequencing, high-performance liquid chromatography-ultraviolet (HPLC-UV), liquid chromatography coupled with tandem mass spectrometry (LC-MS/MS), enzyme-linked immunosorbent assay (ELISA), long interspersed nuclear elements (LINE-1) and Pyrosequencing, amplification fragment length polymorphism (AFLP), restriction fragment length polymorphism (RFLP), luminometric methylation assay, bead-chip, bead-chip array, microarray, methyl-sensitive cut counting, or luminometric methylation.
Embodiment 20. The method of embodiments 14 or 19, further comprising the step of monitoring a methylation status of the one or more CpG biomarkers at one or more points in time and based on a change in methylation discontinuing or restarting treatment with the inhibitor of CD80/86 immune cell costimulation.
Embodiment 21. The method of embodiments 14 or 20, further comprising the step of monitoring a methylation status of the one or more CpG biomarkers at one or more points in time of a patient undergoing treatment with the inhibitor of CD80/86 immune cell costimulation and based on a change in methylation selecting an alternative treatment.
Embodiment 22. A CTLA4/T cell activation methylation signature predicting responsiveness to an inhibitor of CD80/86 immune cell costimulation, the signature comprising:
hypomethylation in a genome of at least one of T cells, B cells, or monocytes in one or more CpG biomarkers selected from;
Cg04612171;
Cg16733676;
Cg02901522; or
Cg11986743;
wherein the hypomethylation at the one or more CpG biomarkers is compared to a subject that does not have lupus in the at least one of the T cells, B cells, and monocytes.
Embodiment 23. The signature of embodiment 22, wherein the inhibitor of CD80/86 immune cell costimulation is Abatacept or Belatacept.
Embodiment 24. The signature of embodiments 22 or 23, wherein the hypomethylation is detected for any 2, 3 or 4 of Cg04612171 and Cg16733676; Cg02901522; and Cg11986743.
Embodiment 25. The signature of embodiments 22 to 24, wherein the hypomethylation is detected for any 2 or 3 of the T cells, B cells, and monocytes.
Embodiment 26. The signature of embodiments 22 to 25, wherein the hypomethylation is detected for any 2 or 3 of the T cells, B cells, and monocytes and is detected for any 2, 3 or 4 of Cg04612171 and Cg16733676; Cg02901522; and Cg11986743.
Embodiment 27. The signature of embodiments 22 to 26, wherein the hypomethylation is detected in T cells and there is an increase in T cell receptor (TCR) signaling genes and a decrease in T cell proliferation.
Embodiment 28. The signature of embodiments 22 to 27, wherein the hypomethylation is detected in monocytes and there is an increase in expression of genes in retinoic acid pathway genes.
Embodiment 29. The signature of embodiments 22 to 28, wherein the hypomethylation is detected in B cells and there is a decrease in B cell receptor (BCR) activity, Fc gamma receptor IIB (FCγRIIB) activity.
Embodiment 30. The signature of embodiments 22 to 29, wherein the hypomethylation is detected by bisulfite sequencing, high-performance liquid chromatography-ultraviolet (hypomethylation-UV), liquid chromatography coupled with tandem mass spectrometry (LC-MS/MS), enzyme-linked immunosorbent assay (ELISA), long interspersed nuclear elements (LINE-1) and Pyrosequencing, amplification fragment length polymorphism (AFLP), restriction fragment length polymorphism (RFLP), luminometric methylation assay, bead-chip, bead-chip array, microarray, methyl-sensitive cut counting, or luminometric methylation.
Embodiment 31. The signature of embodiments 22 to 30, wherein the hypomethylation of Cg04612171 and Cg16733676; Cg02901522; and Cg11986743, affects expression of Sine Oculis Binding Protein Homolog (SOBP), Solute Carrier Family 25 Member 24 (SLC25A24), Plasmacytoma Variant Translocation 1 (PVT1), or Beta-1,4-Galactosyltransferase 6 (B4GALT6), respectively.
Embodiment 32. A method for diagnosing lupus patients responsive to treatment with an inhibitor of CD80/86 immune cell costimulation, comprising:
determining whether a patient has a genetic predisposition to being responsive to the treatment with the inhibitor of CD80/86 immune cell costimulation:
obtaining or having obtained a whole blood sample from the patient with lupus;
separating T cells, B cells, and monocytes;
performing or having performed a methylation assay on a genome of at least one of the T cells, B cells, or monocytes to detect methylation at one or more CpG biomarkers selected from;
Cg04612171;
Cg16733676;
Cg02901522; or
Cg11986743; and
determining if the lupus patient has hypomethylation at the one or more CpG biomarkers when compared to a subject that does not have lupus in the at least one of the T cells, B cells, and monocytes, wherein the lupus is systemic lupus erythematosus, Cutaneous lupus erythematosus, drug-induced lupus, or neonatal lupus, wherein patients with hypomethylation responsive to treatment with the inhibitor of CD80/86 immune cell costimulation.
Embodiment 33. The method of claim 32, wherein the lupus is systemic lupus erythematosus, Cutaneous lupus erythematosus, drug-induced lupus, or neonatal lupus.
Embodiment 34. The method of embodiments 32 or 33, further comprising the step of monitoring a methylation status of the one or more CpG biomarkers at one or more points in time and based on a change in methylation discontinuing or restarting treatment with the inhibitor of CD80/86 immune cell costimulation.
Embodiment 35. The method of any one of embodiments 32 to 34, further comprising the step of monitoring a methylation status of the one or more CpG biomarkers at one or more points in time of a patient undergoing treatment with the inhibitor of CD80/86 immune cell costimulation and based on a change in methylation selecting an alternative treatment.

### REFERENCES

1. Frodlund, M., et al. The majority of Swedish systemic lupus erythematosus patients are still affected by irreversible organ impairment: factors related to damage accrual in two regional cohorts. Lupus 28, 1261-1272 (2019).
2. Borchers, A. T., Keen, C. L., Shoenfeld, Y. & Gershwin, M. E. Surviving the butterfly and the wolf: mortality trends in systemic lupus erythematosus. Autoimmun Rev 3, 423-453 (2004).
3. Rovin, B. H., et al. Efficacy and safety of voclosporin versus placebo for lupus nephritis (AURORA 1): a double-blind, randomised, multicentre, placebo-controlled, phase 3 trial. The Lancet 397, 2070-2080 (2021).
4. Morand, E. F., et al. Trial of Anifrolumab in Active Systemic Lupus Erythematosus. N Engl J Med 382, 211-221 (2020).
5. ClinicalTrials.gov. 1052 Studies found for: lupus.
6. Merrill, J. T. & Buyon, J. P. Connective tissue diseases: What does the death of Riquent hold for the future of SLE? Nat Rev Rheumatol 5, 306-307 (2009).
7. Furie, R. A., et al. A phase 2, randomised, placebo-controlled clinical trial of blisibimod, an inhibitor of B cell activating factor, in patients with moderate-to-severe systemic lupus erythematosus, the PEARL-SC study. Ann Rheum Dis 74, 1667-1675 (2015).
8. Wallace, D. J., et al. Efficacy and safety of epratuzumab in patients with moderate/severe active systemic lupus erythematosus: results from EMBLEM, a phase IIb, randomised, double-blind, placebo-controlled, multicentre study. Ann Rheum Dis 73, 183-190 (2014).
9. Furie, R., et al. A phase III, randomized, placebo-controlled study of belimumab, a monoclonal antibody that inhibits B lymphocyte stimulator, in patients with systemic lupus erythematosus. Arthritis Rheum 63, 3918-3930 (2011).
10. Navarra, S. V., et al. Efficacy and safety of belimumab in patients with active systemic lupus erythematosus: a randomised, placebo-controlled, phase 3 trial. Lancet 377, 721-731 (2011).
11. Merrill, J. T. Emergence of targeted immune therapies for systemic lupus. Expert Opin Emerg Drugs 10, 53-65 (2005).
12. Merrill, J. T., Erkan, D. & Buyon, J. P. Challenges in bringing the bench to bedside in drug development for SLE. Nat Rev Drug Discov 3, 1036-1046 (2004).
13. Bandyopadhyay, S., et al. De-Convolution of Whole Blood Transcriptomic Data from a Phase IIb, Randomized, Double-Blind, Placebo-Controlled Trial of Abatacept in Systemic Lupus Erythematosus. Arthritis & Rheumatology 67, 2463-2464 (2015).
14. Hoffman, R. W., et al. Gene Expression and Pharmacodynamic Changes in 1,760 Systemic Lupus Erythematosus Patients From Two Phase III Trials of BAFF Blockade With Tabalumab. Arthritis Rheumatol 69, 643-654 (2017).
15. Pego-Reigosa, J. M., et al. Analysis of disease activity and response to treatment in a large Spanish cohort of patients with systemic lupus erythematosus. Lupus 24, 720-729 (2015).
16. Baker, K., Pope, J., Fortin, P., Silverman, E. & Peschken, C. Work disability in systemic lupus erythematosus is prevalent and associated with socio-demographic and disease related factors. Lupus 18, 1281-1288 (2009).
17. Marion, T. N. & Postlethwaite, A. E. Chance, genetics, and the heterogeneity of disease and pathogenesis in systemic lupus erythematosus. Semin Immunopathol 36, 495-517 (2014).
18. Merrill, J. T., et al. The Biomarkers of Lupus Disease Study: A Bold Approach May Mitigate Interference of Background Immunosuppressants in Clinical Trials. Arthritis Rheumatol 69, 1257-1266 (2017).
19. Treatment of Lupus Nephritis With Abatacept: The Abatacept and Cyclophosphamide Combination Efficacy and Safety Study. Arthritis & Rheumatology 66, 3096-3104 (2014).
20. Merrill, J. T., et al. The efficacy and safety of abatacept in patients with non-life-threatening manifestations of systemic lupus erythematosus: results of a twelve-month, multicenter, exploratory, phase IIb, randomized, double-blind, placebo-controlled trial. Arthritis Rheum 62, 3077-3087 (2010).
21. Furie, R., et al. Efficacy and safety of abatacept in lupus nephritis: a twelve-month, randomized, double-blind study. Arthritis Rheumatol 66, 379-389 (2014).
22. Bandyopadhyay, S., et al. Identification of biomarkers of response to abatacept in patients with SLE using deconvolution of whole blood transcriptomic data from a phase IIb clinical trial. Lupus Sci Med 4, e000206 (2017).
23. Hedrich, C. M., Mäbert, K., Rauen, T. & Tsokos, G. C. DNA methylation in systemic lupus erythematosus. Epigenomics 9, 505-525 (2017).
24. Coit, P., et al. Hypomethylation of miR-17-92 cluster in lupus T cells and no significant role for genetic factors in the lupus-associated DNA methylation signature. Annals of the Rheumatic Diseases 81, 1428-1437 (2022).
25. Renauer, P., et al. DNA methylation patterns in naïve CD4+ T cells identify epigenetic susceptibility loci for malar rash and discoid rash in systemic lupus erythematosus. Lupus Science and Medicine 2 (2015).
26. Jeffries, M. A., et al. Genome-wide DNA methylation patterns in CD4+ T cells from patients with systemic lupus erythematosus. Epigenetics 6, 593-601 (2011).
27. Coit, P., et al. Genome-wide DNA methylation study suggests epigenetic accessibility and transcriptional poising of interferon-regulated genes in naïve CD4+ T cells from lupus patients. J Autoimmun 43, 78-84 (2013).
28. Absher, D. M., et al. Genome-wide DNA methylation analysis of systemic lupus erythematosus reveals persistent hypomethylation of interferon genes and compositional changes to CD4+ T-cell populations. PLOS Genet 9, e1003678 (2013).
29. Coit, P., et al. Epigenome profiling reveals significant DNA demethylation of interferon signature genes in lupus neutrophils. J Autoimmun 58, 59-66 (2015).
30. Banchereau, R., et al. Personalized Immunomonitoring Uncovers Molecular Networks that Stratify Lupus Patients. Cell 165, 551-565 (2016).
31. Guthridge, J. M., et al. Adults with systemic lupus exhibit distinct molecular phenotypes in a cross-sectional study. EClinicalMedicine 20, 100291 (2020).
32. Perez, R. K., et al. Single-cell RNA-seq reveals cell type&#x2013; specific molecular and genetic associations to lupus. Science 376, eabf1970 (2022).
33. Andreoletti, G., et al. Transcriptomic analysis of immune cells in a multi-ethnic cohort of systemic lupus erythematosus patients identifies ethnicity- and disease-specific expression signatures. Communications Biology 4, 488 (2021).
34. Nehar-Belaid, D., et al. Mapping systemic lupus erythematosus heterogeneity at the single-cell level. Nat Immunol 21, 1094-1106 (2020).
35. Huang, X., et al. Multi-Platform Omics Analysis Reveals Molecular Signatures for Pathogenesis and Activity of Systemic Lupus Erythematosus. Front Immunol 13, 833699 (2022).
36. Thanou, A., Jupe, E., Purushothaman, M., Niewold, T. B. & Munroe, M. E. Clinical disease activity and flare in SLE: Current concepts and novel biomarkers. J Autoimmun 119, 102615 (2021).
37. Zhao, M., et al. IFI44L promoter methylation as a blood biomarker for systemic lupus erythematosus. Ann Rheum Dis 75, 1998-2006 (2016).
38. Mok, A., et al. Genome-wide profiling identifies associations between lupus nephritis and differential methylation of genes regulating tissue hypoxia and type 1 interferon responses. Lupus Sci Med 3, e000183 (2016).
39. Coit, P., et al. Renal involvement in lupus is characterized by unique DNA methylation changes in naïve CD4+ T cells. Journal of Autoimmunity 61, 29-35 (2015).
40. Der, E., et al. Tubular cell and keratinocyte single-cell transcriptomics applied to lupus nephritis reveal type I IFN and fibrosis relevant pathways. Nature Immunology 20, 915-927 (2019).
41. Shipa, M., et al. Identification of biomarkers to stratify response to B-cell-targeted therapies in systemic lupus erythematosus: an exploratory analysis of a randomised controlled trial. The Lancet Rheumatology 5, e24-e35 (2023).
42. Isenberg, D. A., et al. BILAG 2004. Development and initial validation of an updated version of the British Isles Lupus Assessment Group's disease activity index for patients with systemic lupus erythematosus. Rheumatology 44, 902-906 (2005).
43. Petri, M., Genovese, M., Engle, E. & Hochberg, M. Definition, incidence, and clinical description of flare in systemic lupus erythematosus. A prospective cohort study. Arthritis & Rheumatism 34, 937-944 (1991).
44. Gladman, D. D., Ibañez, D. & Urowitz, M. B. Systemic lupus erythematosus disease activity index 2000. J Rheumatol 29, 288-291 (2002).
45. Buyon, J. P., et al. The effect of combined estrogen and progesterone hormone replacement therapy on disease activity in systemic lupus erythematosus: a randomized trial. Ann Intern Med 142, 953-962 (2005).
46. Thanou, A., et al. Scoring systemic lupus erythematosus (SLE) disease activity with simple, rapid outcome measures. Lupus Sci Med 6, e000365 (2019).
47. Asare, A. L., Carey, V. J., Rotrosen, D. & Nepom, G. T. Clinical trial data access: Opening doors with TrialShare. J Allergy Clin Immunol 138, 724-726 (2016).
48. Di Tommaso, P., et al. Nextflow enables reproducible computational workflows. Nature Biotechnology 35, 316-319 (2017).
49. Patro, R., Duggal, G., Love, M. I., Irizarry, R. A. & Kingsford, C. Salmon provides fast and bias-aware quantification of transcript expression. Nature Methods 14, 417-419 (2017).
50. Frankish, A., et al. GENCODE reference annotation for the human and mouse genomes. Nucleic Acids Research 47, D766-D773 (2018).
51. Team, R. C. R: A Language and Environment for Statistical Computing, (R Foundation for Statistical Computing).
52. McCarthy, D. J., Chen, Y. & Smyth, G. K. Differential expression analysis of multifactor RNA-Seq experiments with respect to biological variation. Nucleic Acids Res 40, 4288-4297 (2012).
53. Ritchie, M. E., et al. limma powers differential expression analyses for RNA-sequencing and microarray studies. Nucleic Acids Res 43, e47 (2015).
54. Fortin, J. P., Triche, T. J., Jr. & Hansen, K. D. Preprocessing, normalization and integration of the Illumina HumanMethylationEPIC array with minfi. Bioinformatics 33, 558-560 (2017).
55. Friedman, J., Hastie, T. & Tibshirani, R. Regularization Paths for Generalized Linear Models via Coordinate Descent. J Stat Softw 33, 1-22 (2010).
56. Sing, T., Sander, O., Beerenwinkel, N. & Lengauer, T. ROCR: visualizing classifier performance in R. Bioinformatics 21, 3940-3941 (2005).
57. Phipson, B., Maksimovic, J. & Oshlack, A. missMethyl: an R package for analyzing data from Illumina's HumanMethylation450 platform. Bioinformatics 32, 286-288 (2016).
58. Thanou, A., et al. Abatacept Failed to Demonstrate Efficacy in an SLE Trial with Low Placebo Response Rates, Although Global Assessments Indicated Less Flare Severity [abstract]. Arthritis Rheumatol 71 (2019).
59. Chen, Z., et al. Jxc1/Sobp, encoding a nuclear zinc finger protein, is critical for cochlear growth, cell fate, and patterning of the organ of corti. J Neurosci 28, 6633-6641 (2008).
60. Birk, E., et al. SOBP is mutated in syndromic and nonsyndromic intellectual disability and is highly expressed in the brain limbic system. Am J Hum Genet 87, 694-700 (2010).
61. Tavares, A. L. P., Jourdeuil, K., Neilson, K. M., Majumdar, H. D. & Moody, S. A. Sobp modulates the transcriptional activation of Six1 target genes and is required during craniofacial development. Development 148 (2021).
62. Lei, J., et al. Identification of MEG8/miR-378d/SOBP axis as a novel regulatory network and associated with immune infiltrates in ovarian carcinoma by integrated bioinformatics analysis. Cancer Med 10, 2924-2939 (2021).
63. Aprille, J. R. Mechanism and regulation of the mitochondrial ATP-Mg/P (i) carrier. J Bioenerg Biomembr 25, 473-481 (1993).
64. Fiermonte, G., et al. Identification of the mitochondrial ATP-Mg/Pi transporter. Bacterial expression, reconstitution, functional characterization, and tissue distribution. J Biol Chem 279, 30722-30730 (2004).
65. Writzl, K., et al. De Novo Mutations in SLC25A24 Cause a Disorder Characterized by Early Aging, Bone Dysplasia, Characteristic Face, and Early Demise. Am J Hum Genet 101, 844-855 (2017).
66. Hong, T., et al. Epstein-Barr virus nuclear antigen 2 extensively rewires the human chromatin landscape at autoimmune risk loci. Genome Res 31, 2185-2198 (2021).
67. Zhao, L., et al. Mitochondrial impairment and repair in the pathogenesis of systemic lupus erythematosus. Front Immunol 13, 929520 (2022).
68. Ali, M. A., et al. Serum long noncoding RNAs FAS-AS1 & PVT1 are novel biomarkers for systemic lupus erythematous. Br J Biomed Sci 77, 208-212 (2020).
69. Liu, C.-X., et al. Structure and Degradation of Circular RNAs Regulate PKR Activation in Innate Immunity. Cell 177, 865-880.e821 (2019).
70. Li, X., et al. Circular RNA circPVT1 Promotes Proliferation and Invasion Through Sponging miR-125b and Activating E2F2 Signaling in Non-Small Cell Lung Cancer. Cell Physiol Biochem 51, 2324-2340 (2018).
71. Mayo, L., et al. Regulation of astrocyte activation by glycolipids drives chronic CNS inflammation. Nat Med 20, 1147-1156 (2014).
72. Coit, P., et al. A longitudinal and transancestral analysis of DNA methylation patterns and disease activity in lupus patients. JCI Insight 5 (2020).
73. Nestor, C. E., et al. 5-Hydroxymethylcytosine Remodeling Precedes Lineage Specification during Differentiation of Human CD4<sup>+</sup> T Cells. Cell Reports 16, 559-570 (2016).
74. Zhang, Z., et al. Comparative analysis of the DNA methylation landscape in CD4, CD8, and B memory lineages. Clinical Epigenetics 14, 173 (2022).
75. Wu, H., Zhao, M., Tan, L. & Lu, Q. The key culprit in the pathogenesis of systemic lupus erythematosus: Aberrant DNA methylation. Autoimmun Rev 15, 684-689 (2016).

1. A method to select lupus patients that will be responsive to a treatment with an inhibitor of CD80/86 immune cell costimulation, comprising:
determining whether a patient has a genetic predisposition to being responsive to the treatment with the inhibitor of CD80/86 immune cell costimulation by:
obtaining or having obtained a whole blood sample from the patient with lupus;
separating T cells, B cells, and monocytes;
performing or having performed a methylation assay on a genome of at least one of the T cells, B cells, or monocytes to detect methylation at one or more CpG biomarkers selected from;
Cg04612171;
Cg16733676;
Cg02901522; or
Cg11986743;
determining if the patient has methylation at the one or more CpG biomarkers when compared to a subject that does not have lupus in at least one of the T cells, B cells, or monocytes, and
administering the inhibitor of CD80/86 immune cell costimulation if the patient has methylation at the one or more of the CpG biomarkers in the at least one of the T cells, B cells, or monocytes.

2. The method of claim 1, wherein the inhibitor of CD80/86 immune cell costimulation is Abatacept or Belatacept.

3. The method of claim 1, wherein the methylation is detected for any 2, 3 or 4 of Cg04612171, Cg16733676, Cg02901522, or Cg11986743.

4. The method of claim 1, wherein the methylation is detected in at least one of:
2 or 3 of the T cells, B cells, or monocytes;
2 or 3 of the T cells, B cells, or monocytes and is detected at any 2, 3 or 4 of Cg04612171, Cg16733676, Cg02901522, or Cg11986743;
the T cells and there is an increase in regulation of T cell receptor (TCR) signaling genes, a decrease in T cell proliferation, or both;
the monocytes and there is an increase in expression of genes in retinoic acid pathway genes; or
the B cells and there is a decrease in B cell receptor (BCR) activity, Fc gamma receptor IIB (FCγRIIB) activity, or both.

5. (canceled)

6. (canceled)

7. (canceled)

8. (canceled)

9. The method of claim 1, wherein the methylation is detected by bisulfite sequencing, high-performance liquid chromatography-ultraviolet (HPLC-UV), liquid chromatography coupled with tandem mass spectrometry (LC-MS/MS), enzyme-linked immunosorbent assay (ELISA), long interspersed nuclear elements (LINE-1) and Pyrosequencing, amplification fragment length polymorphism (AFLP), restriction fragment length polymorphism (RFLP), luminometric methylation assay, bead-chip, bead-chip array, microarray, methyl-sensitive cut counting, or luminometric methylation.

10. The method of claim 1, wherein the methylation of Cg04612171, Cg16733676, Cg02901522, and Cg11986743, affects expression of Sine Oculis Binding Protein Homolog (SOBP), Solute Carrier Family 25 Member 24 (SLC25A24), Plasmacytoma Variant Translocation 1 (PVT1), and Beta-1,4-Galactosyltransferase 6 (B4GALT6), respectively.

11. The method of claim 1, wherein the lupus is systemic lupus erythematosus, Cutaneous lupus erythematosus, drug-induced lupus, or neonatal lupus.

12. The method of claim 1, further comprising at least one of:
the step of monitoring a methylation status of the one or more CpG biomarkers at one or more points in time and based on a change in methylation discontinuing or restarting treatment with the inhibitor of CD80/86 immune cell costimulation; or
the step of monitoring a methylation status of the one or more CpG biomarkers at one or more points in time of a patient undergoing treatment with the inhibitor of CD80/86 immune cell costimulation and based on a change in methylation selecting an alternative treatment.

13. (canceled)

14. A method of treating lupus patients that will be responsive for a treatment with an inhibitor of CD80/86 immune cell costimulation, comprising:
determining whether a patient has a genetic predisposition to being responsive to the treatment with the inhibitor of CD80/86 immune cell costimulation:
obtaining or having obtained a whole blood sample from the patient with lupus;
separating T cells, B cells, and monocytes;
performing or having performed a methylation assay on a genome of at least one of the T cells, B cells, or monocytes to detect methylation at one or more CpG biomarkers selected from;
Cg04612171;
Cg16733676;
Cg02901522; or
Cg11986743;
determining if the lupus patient has methylation at the one or more CpG biomarkers when compared to a subject that does not have lupus in at least one of the T cells, B cells, or monocytes, wherein the lupus is systemic lupus erythematosus, Cutaneous lupus erythematosus, drug-induced lupus, or neonatal lupus; and
administering the inhibitor of CD80/86 immune cell costimulation if the patient has methylation of the one or more of the CpG biomarkers in at least one of the T cells, B cells, or monocytes; or
administering to the patient that does not have methylation of the one or more of the CpG biomarkers in at least one of the T cells, B cells, or monocytes with at least one of NSAIDs, corticosteroids, immunosuppressants, hydroxychloroquine, or methotrexate.

15. The method of claim 14, wherein the inhibitor of CD80/86 immune cell costimulation is Abatacept or Belatacept.

16. (canceled)

17. The method of claim 14, wherein the methylation is detected in at least one of:
2 or 3 of the T cells, B cells, or monocytes;
2 or 3 of the T cells, B cells, or monocytes and is detected for any 2, 3 or 4 of Cg04612171, Cg16733676, Cg02901522, or Cg11986743;
the T cells and there is an increase in regulation of T cell receptor (TCR) signaling genes a decrease in T cell proliferation, or both;
the monocytes and there is an increase in expression of genes in retinoic acid pathway genes; or
the B cells and there is a decrease in B cell receptor (BCR) activity, Fc gamma receptor IIB (FCγRIIB) activity, or both.

18. (canceled)

19. The method of claim 14, wherein the methylation is detected by bisulfite sequencing, high-performance liquid chromatography-ultraviolet (HPLC-UV), liquid chromatography coupled with tandem mass spectrometry (LC-MS/MS), enzyme-linked immunosorbent assay (ELISA), long interspersed nuclear elements (LINE-1) and Pyrosequencing, amplification fragment length polymorphism (AFLP), restriction fragment length polymorphism (RFLP), luminometric methylation assay, bead-chip, bead-chip array, microarray, methyl-sensitive cut counting, or luminometric methylation.

20. The method of claim 14, further comprising at least one of:
the step of monitoring a methylation status of the one or more CpG biomarkers at one or more points in time and based on a change in methylation discontinuing or restarting treatment with the inhibitor of CD80/86 immune cell costimulation; or
the step of monitoring a methylation status of the one or more CpG biomarkers at one or more points in time of a patient undergoing treatment with the inhibitor of CD80/86 immune cell costimulation and based on a change in methylation selecting an alternative treatment.

21. (canceled)

22. A CTLA4/T cell activation methylation signature predicting responsiveness to an inhibitor of CD80/86 immune cell costimulation, the signature comprising:
methylation in a genome of at least one of T cells, B cells, or monocytes in one or more CpG biomarkers selected from;
Cg04612171;
Cg16733676;
Cg02901522; or
Cg11986743;
wherein the methylation at the one or more CpG biomarkers is compared to a subject that does not have lupus in at least one of the T cells, B cells, or monocytes.

23. The signature of claim 22, wherein the inhibitor of CD80/86 immune cell costimulation is Abatacept or Belatacept.

24. The signature of claim 22, wherein the methylation is detected in at least one of:
2 or 3 of the T cells, B cells, or monocytes;
2 or 3 of the T cells, B cells, or monocytes and is detected for any 2, 3 or 4 of Cg04612171, Cg16733676, Cg02901522, or Cg11986743;
the T cells and there is an increase in regulation of T cell receptor (TCR) signaling genes, a decrease in T cell proliferation, or both;
the monocytes and there is an increase in expression of genes in retinoic acid pathway genes; or
the B cells and there is a decrease in B cell receptor (BCR) activity, Fc gamma receptor IIB (FCγRIIB) activity, or both.

25. The signature of claim 22, wherein the methylation is detected for any 2 or 3 of the T cells, B cells, or monocytes.

26. (canceled)

27. (canceled)

28. (canceled)

29. (canceled)

30. The signature of claim 22, wherein the methylation is detected by bisulfite sequencing, high-performance liquid chromatography-ultraviolet, liquid chromatography coupled with tandem mass spectrometry (LC-MS/MS), enzyme-linked immunosorbent assay (ELISA), long interspersed nuclear elements (LINE-1) and Pyrosequencing, amplification fragment length polymorphism (AFLP), restriction fragment length polymorphism (RFLP), luminometric methylation assay, bead-chip, bead-chip array, microarray, methyl-sensitive cut counting, or luminometric methylation.

31. The signature of claim 22, wherein the methylation of Cg04612171, Cg16733676, Cg02901522, and Cg11986743, affects expression of Sine Oculis Binding Protein Homolog (SOBP), Solute Carrier Family 25 Member 24 (SLC25A24), Plasmacytoma Variant Translocation 1 (PVT1), and Beta-1,4-Galactosyltransferase 6 (B4GALT6), respectively.

32. A method for diagnosing lupus patients responsive to treatment with an inhibitor of CD80/86 immune cell costimulation, comprising:
determining whether a patient has a genetic predisposition to being responsive to the treatment with the inhibitor of CD80/86 immune cell costimulation:
obtaining or having obtained a whole blood sample from the patient with lupus;
separating T cells, B cells, and monocytes;
performing or having performed a methylation assay on a genome of at least one of the T cells, B cells, or monocytes to detect methylation at one or more CpG biomarkers selected from;
Cg04612171;
Cg16733676;
Cg02901522; or
Cg11986743; and
determining if the lupus patient has methylation at the one or more CpG biomarkers when compared to a subject that does not have lupus in at least one of the T cells, B cells, or monocytes, wherein the lupus is systemic lupus erythematosus, Cutaneous lupus erythematosus, drug-induced lupus, or neonatal lupus, wherein patients with methylation at the one or more CpG biomarkers in at least one of T cells, B cells, or monocytes are responsive to treatment with the inhibitor of CD80/86 immune cell costimulation.

33. (canceled)

34. The method of claim 32, further comprising at least one of:
the step of monitoring a methylation status of the one or more CpG biomarkers at one or more points in time and based on a change in methylation discontinuing or restarting treatment with the inhibitor of CD80/86 immune cell costimulation; or the step of monitoring a methylation status of the one or more CpG biomarkers at one or more points in time of a patient undergoing treatment with the inhibitor of CD80/86 immune cell costimulation and based on a change in methylation selecting an alternative treatment.

35. (canceled)
